SEQUENCE LISTING (37 sequences)

SEQ ID NO: 1 - DNA, 73 bp, Artificial Sequence
  zymogene/primer
ttgacgatacagctgccaccagggcctagctacaacgatccaactaccacaagtcagaagcttccaaatcctg

SEQ ID NO: 2 - DNA, 19 bp, Artificial Sequence
  primer
aagtgggcatttgaacaac

SEQ ID NO: 3 - DNA, 15 bp, Artificial Sequence
  catalytic nucleic acid
ggctagctacaacga

SEQ ID NO: 4 - DNA, 14 bp, Artificial Sequence
  catalytic nucleic acid
tccgagccggacga

SEQ ID NO: 5 - DNA, 13 bp, Artificial Sequence
  catalytic nucleic acid
ccgagccggacga

SEQ ID NO: 6 - DNA, 29 bp, Artificial Sequence
  catalytic nucleic acid
catatactccgagccggacgacacgtcgc

SEQ ID NO: 7 - DNA, 14 bp, Artificial Sequence
  catalytic nucleic acid
gtcagcgacacgaa

SEQ ID NO: 8 - DNA, 33 bp, Artificial Sequence
  catalytic nucleic acid
gctgttgatctgtcagcgacacgaaatggtgat

SEQ ID NO: 9 - DNA, 42 bp, Artificial Sequence
  catalytic nucleic acid
ggagaacgcgaggcaaggctgggagaaatgtggatcacgatt

SEQ ID NO: 10 - DNA, 61 bp, Artificial Sequence
  catalytic nucleic acid
ggagaacgcgaggcaaggctgggagaaatgtggatcacgattgtctgaatcggtctgtatc

SEQ ID NO: 11 - DNA, 16 bp, Artificial Sequence
  catalytic nucleic acid
acgtggaggtgggctc

SEQ ID NO: 12 - DNA, 33 bp, Artificial Sequence
  catalytic nucleic acid
catctcttctccgagccggtcgaaatagtgagt

SEQ ID NO: 13 - DNA, 24 bp, Artificial Sequence
  catalytic nucleic acid
agcgatccggaacggcacccatgt

SEQ ID NO: 14 - DNA, 15 bp, Artificial Sequence
  catalytic nucleic acid
ggctagctacaacga

SEQ ID NO: 15 - DNA, 25 bp, Artificial Sequence
  catalytic nucleic acid
aacggggctgtgcggctaggaagta

SEQ ID NO: 16 - DNA, 15 bp, Artificial Sequence
  catalytic nucleic acid
gaagtagcgccgccg

SEQ ID NO: 17 - DNA, 14 bp, Artificial Sequence
  catalytic nucleic acid
ccgagccggtcgaa

SEQ ID NO: 18 - DNA, 20 bp, Artificial Sequence
  primer
ctcccaagaatcatggactg

SEQ ID NO: 19 - DNA, 20 bp, Artificial Sequence
  primer
aaagcagagctctcactggt

SEQ ID NO: 20 - DNA, 19 bp, Artificial Sequence
  primer
acccctaaggccaaccgtg

SEQ ID NO: 21 - DNA, 22 bp, Artificial Sequence
  primer
caggattccatacccaagaagg

SEQ ID NO: 22 - DNA, 18 bp, Artificial Sequence
  primer
ggcaagacaatcattgaa

SEQ ID NO: 23 - DNA, 21 bp, Artificial Sequence
  primer
ggttgagttcacttatttgaa

SEQ ID NO: 24 - DNA, 20 bp, Artificial Sequence
  primer
atgacctatagactctttga

SEQ ID NO: 25 - DNA, 20 bp, Artificial Sequence
  primer
ctgtcacacaagttactaga

SEQ ID NO: 26 - DNA, 18 bp, Artificial Sequence
  primer
ttttgccagtctccgaat

SEQ ID NO: 27 - DNA, 22 bp, Artificial Sequence
  primer
tgctttatggaaaatttattgc

SEQ ID NO: 28 - DNA, 19 bp, Artificial Sequence
  primer
ggtctgtgatgcccttaga

SEQ ID NO: 29 - DNA, 19 bp, Artificial Sequence
  primer
agcttatgacccgcactta

SEQ ID NO: 30 - DNA, 19 bp, Artificial Sequence
  primer
cgacctggaagtccaacta

SEQ ID NO: 31 - DNA, 20 bp, Artificial Sequence
  primer
ttcggataatcatccaatag

SEQ ID NO: 32 - DNA, 19 bp, Artificial Sequence
  primer
caacggatttggtcgtatt

SEQ ID NO: 33 - DNA, 19 bp, Artificial Sequence
  primer
ttgatggcaacaatatcca

SEQ ID NO: 34 - DNA, 19 bp, Artificial Sequence
  primer
tcgcgctactctctctttc

SEQ ID NO: 35 - DNA, 19 bp, Artificial Sequence
  primer
atctttggagtacgctgga

SEQ ID NO: 36 - DNA, 20 bp, Artificial Sequence
  primer
atgtgtcatccatatttctc

SEQ ID NO: 37 - DNA, 20 bp, Artificial Sequence
  primer
ccacagcagtaattaagatt

(19) United States
(12) Patent Application Publication
(10) Pub. No.: US 2005/0250134 A1
(43) Pub. Date: Nov. 10, 2005

(54) Fluorescent energy transfer labeled nucleic acid substrates and methods of use thereof

(76) Inventors: Dawei Sheng, Mountain View, CA (US); Marcia Tan, Palo Alto, CA (US); Robert D. Larsen, Alameda, CA (US)

Correspondence Address: BOZICEVIC, FIELD & FRANCIS (BD BIOSCIENCES), 1900 UNIVERSITY AVENUE, SUITE 200, EAST PALO ALTO, CA, 94303, US

(21) Appl. No.: 11/070,871

(22) Filed: Mar. 1, 2005

Related U.S. Application Data
(60) Provisional application No. 60/550,637, filed on Mar. 3, 2004.
(60) Provisional application No. 60/549,743, filed on Mar. 2, 2004.

(51) Int. Cl. C12Q 1/68

(52) U.S. Cl. 435/6

(57) ABSTRACT

Methods and compositions for detecting a target nucleic acid in a sample are provided. Also provided are methods and compositions for detecting a plurality of target nucleic acids in a sample. Sets of components for use in a subject method are provided; as well as compositions that include a subject set. The invention further provides kits and systems for practicing the subject methods

### FIELD OF THE INVENTION

The present invention is in the field of nucleic acid amplification reactions, and methods of detecting target nucleic acids.

### BACKGROUND OF THE INVENTION

Methods of in vitro nucleic acid amplification have wide-spread applications in genetics, disease diagnosis, and forensics. In the last decade, many techniques for amplification of known nucleic acid sequences (“targets”) have been described. These include the polymerase chain reaction (“PCR”), the strand displacement amplification assay (“SDA”) and transcription-mediated amplification (“TMA”) (also known as self-sustained sequence replication (“SSR”)). The amplification products (“amplicons”) produced by PCR and SDA are DNA, whereas RNA amplicons are produced by TMA. The DNA or RNA amplicons generated by these methods can be used as markers of nucleic acid sequences associated with specific disorders.

Several methods allow simultaneous amplification and detection of nucleic acids in a closed system, i.e., in a single homogeneous reaction system. These methods include Sunrise™ primer-based systems, Molecular Beacons, the Taqman™ system, an Amplifluor™ hairpin primer-based system, a Scorpions technology (e.g., bi-functional molecules containing a PCR primer element covalently linked to a probe element), a Light Upon Extension or LUX™-based system, and detection systems based on use of the fluorescent dye SYBR Green. Using homogeneous sealed tube formats has several advantages over separately analyzing amplicons following amplification reactions. Closed system methods are faster and simpler because they require fewer manipulations. A closed system eliminates the potential for false positives associated with contamination by amplicons from other reactions. Homogeneous reactions can be monitored in real time, with the signal at time zero allowing the measurement of the background signal in the system. Additional control reactions for estimating the background signal are therefore not required. A change in the signal intensity indicates amplification of a specific nucleic acid sequence present in the sample.

Despite the numerous detection chemistries for real-time detection of PCR products, there continues to be interest in the development of new assays capable of providing certain advantages over that which has already been developed.

Literature

U.S. Pat. Nos. 6,008,373, 6,140,055, 6,174,670, 6,201,113, 6,361,941, and 6,365,724; and WO 03/040397.

### SUMMARY OF THE INVENTION

Methods and compositions for detecting a target nucleic acid in a sample are provided. Also provided are methods and compositions for detecting a plurality of target nucleic acids in a sample. Sets of components for use in a subject method are provided; as well as compositions that include a subject set. The invention further provides kits and systems for practicing the subject methods.

### BRIEF DESCRIPTION OF THE DRAWINGS

FIG. 1 depicts an exemplary FET-labeled substrate. R1 and R2, substrate recognition domains; CS, cleavage site in the substrate.

FIG. 2 depicts an exemplary embodiment of a subject amplification reaction. R1 and R2 are substrate recognition domains. C, catalytic domain; BHQ, Black Hole Quencher dye. DNA strands are labeled as follows: (1), target nucleic acid; (2), first, enzymatically inactive amplification product; (3) second, enzymatically active amplification product.

FIG. 3 depicts an exemplary embodiment of a subject amplification reaction, adapted for analysis of two target nucleic acids (“duplex analysis”). The first target nucleic acid includes a nucleotide sequence of a glyceraldehydes 3-phosphate dehydrogenase (GAPDH) gene; and the second target nucleic acid includes a nucleotide sequence of a ribosomal protein L15 (RPLO) gene.

FIGS. 4A and 4B depict an exemplary embodiment of a subject amplification reaction, adapted for analysis of three target nucleic acids (“triplex analysis”). The first target nucleic acid includes a nucleotide sequence of a first gene (Gene 1); the second target nucleic acid includes a nucleotide sequence of a second gene (Gene 2); and the third target nucleic acid includes a nucleotide sequence of a third gene (Gene 3).

FIGS. 5A-I depict triplex analysis carried out with a subject method.

FIGS. 6A and 6B depict increased efficiency using a 5′ driver primer according to a subject method.

### DEFINITIONS

The terms “catalytic nucleic acid molecule,” “catalytic nucleic acid,” and “catalytic nucleic acid sequence” are used interchangeably herein, and refer to a DNA molecule or DNA-containing molecule (also known in the art as a “DNAzyme”) or an RNA or RNA-containing molecule (also known in the art as a “ribozyme”) which specifically recognizes a distinct substrate and catalyzes the chemical modification of this substrate. The nucleic acid bases in the DNAzymes and ribozymes can be the bases A, C, G, T and U, as well as derivatives thereof. Derivatives of these bases are well known in the art, and are exemplified in various publications, including, e.g., PCR Systems Reagents and Consumables Perkin Elmer Catalog 1996-1997. Roche Molecular Systems, Inc. Branchburg, N.J., USA.

“Amplification” of a target nucleic acid sequence refers to the exponential amplification thereof (as opposed to linear amplification), whereby each amplification cycle doubles (or nearly doubles) the number of target amplicons present in the immediately preceding the cycle. Methods of exponential amplification include, but are not limited to, PCR, SDA and TMA. Exponential amplification differs from linear amplification; in linear amplification, each amplification cycle increases by a fixed number the number of target amplicons present in the immediately preceding the cycle.

The terms “reporter substrate,” “chemical substrate” and “substrate” are used interchangeably herein, and refer to any molecule which is specifically recognized and modified by a catalytic nucleic acid molecule. “Target” and “target nucleic acid sequence” are equivalent, and each shall mean the nucleic acid sequence of interest to be detected or measured by the instant invention, which comprises a sequence that hybridizes with the primer when contacted therewith in this method, and that can be either an entire molecule or a portion thereof. “Primer” refers to a short segment of DNA or DNA-containing nucleic acid molecule, which (i) anneals under amplification conditions to a suitable portion of a DNA or RNA sequence to be amplified, and (ii) initiates, and is itself physically extended, via polymerase-mediated synthesis. Finally, “zymogene” refers to a nucleic acid sequence which comprises the anti-sense (i.e. complementary) sequence of a catalytic nucleic acid molecule having detectable activity, and whose transcription product is the catalytic nucleic acid molecule.

As used herein, “nucleic acid” refers to either DNA or RNA, single-stranded or double-stranded, and any chemical modifications thereof. Modifications include, but are not limited to, those which provide other chemical groups that incorporate additional charge, polarizability, hydrogen bonding, electrostatic interaction, and functionality to the nucleic acid. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

As used herein, “fluorescent group” refers to a molecule that, when excited with light having a selected wavelength, emits light of a different wavelength. Fluorescent groups may also be referred to as “fluorophores.”

As used herein, “fluorescence-modifying group” refers to a molecule that can alter in any way the fluorescence emission from a fluorescent group. A fluorescence-modifying group generally accomplishes this through an energy transfer mechanism. Depending on the identity of the fluorescence-modifying group, the fluorescence emission can undergo a number of alterations, including, but not limited to, attenuation, complete quenching, enhancement, a shift in wavelength, a shift in polarity, a change in fluorescence lifetime. One example of a fluorescence-modifying group is a quenching group.

As used herein, “energy transfer” refers to the process by which the fluorescence emission of a fluorescent group is altered by a fluorescence-modifying group. If the fluorescence-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena. Energy transfer is also referred to herein as fluorescent energy transfer or FET.

As used herein, “energy transfer pair” refers to any two molecules that participate in energy transfer. Typically, one of the molecules acts as a fluorescent group, and the other acts as a fluorescence-modifying group. Where the energy transfer pair comprises a fluorescent group and a fluorescence-modifying group, the energy transfer pair is also referred to herein as a “fluorescent energy transfer pair.” An exemplary energy transfer pair comprises a fluorescent group and a quenching group. In some cases, the distinction between the fluorescent group and the fluorescence-modifying group may be blurred. For example, under certain circumstances, two adjacent fluorescein groups can quench one another's fluorescence emission via direct energy transfer. For this reason, there is no limitation on the identity of the individual members of the energy transfer pair in this application. All that is required is that the spectroscopic properties of the energy transfer pair as a whole change in some measurable way if the distance between the individual members is altered by some critical amount.

“Energy transfer pair” is used to refer to a group of molecules that form a single complex within which energy transfer occurs. Such complexes may comprise, for example, two fluorescent groups which may be different from one another and one quenching group, two quenching groups and one fluorescent group, or multiple fluorescent groups and multiple quenching groups. In cases where there are multiple fluorescent groups and/or multiple quenching groups, the individual groups may be different from one another.

As used herein, “quenching group” refers to any fluorescence-modifying group that can attenuate at least partly the light emitted by a fluorescent group. We refer herein to this attenuation as “quenching”. Hence, illumination of the fluorescent group in the presence of the quenching group leads to an emission signal that is less intense than expected, or even completely absent. Quenching occurs through energy transfer between the fluorescent group and the quenching group.

As used herein, “fluorescence resonance energy transfer” or “FRET” refers to an energy transfer phenomenon in which the light emitted by the excited fluorescent group is absorbed at least partially by a fluorescence-modifying group. If the fluorescence-modifying group is a quenching group, then that group can either radiate the absorbed light as light of a different wavelength, or it can dissipate it as heat. FRET depends on an overlap between the emission spectrum of the fluorescent group and the absorption spectrum of the quenching group. FRET also depends on the distance between the quenching group and the fluorescent group. Above a certain critical distance, the quenching group is unable to absorb the light emitted by the fluorescent group, or can do so only poorly.

As used herein “direct energy transfer” refers to an energy transfer mechanism in which passage of a photon between the fluorescent group and the fluorescence-modifying group does not occur. Without being bound by a single mechanism, it is believed that in direct energy transfer, the fluorescent group and the fluorescence-modifying group interfere with each others electronic structure. If the fluorescence-modifying group is a quenching group, this will result in the quenching group preventing the fluorescent group from even emitting light.

In general, quenching by direct energy transfer is more efficient than quenching by FRET. Indeed, some quenching groups that do not quench particular fluorescent groups by FRET (because they do not have the necessary spectral overlap with the fluorescent group) can do so efficiently by direct energy transfer. Furthermore, some fluorescent groups can act as quenching groups themselves if they are close enough to other fluorescent groups to cause direct energy transfer. For example, under these conditions, two adjacent fluorescein groups can quench one another's fluorescence effectively. For these reasons, there is no limitation on the nature of the fluorescent groups and quenching groups useful for the practice of this invention.

As used herein, “3′ end” means at any location on the oligonucleotide from and including the 3′ terminus to the center of the oligonucleotide, usually at any location from and including the 3′ terminus to about 10 bp from the 3′ terminus, and more usually at any location from and including the 3′ terminus to about 5 bp from the 3′ terminus.

As used herein, “5′ end” means at any location on the oligonucleotide from and including the 5′ terminus to the center of the oligonucleotide, usually at any location from and including the 5′ terminus to about 10 bp from the 5′ terminus, and more usually at any location from and including the 5′ terminus to about 5 bp from the 5′ terminus.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as nucleated cells, CD4⁺ T lymphocytes, glial cells, macrophages, tumor cells, peripheral blood mononuclear cells (PBMC), and the like; enrichment for particular cell fractions, e.g., nuclei, mitochondria, etc.; enrichment for particular macromolecules, e.g., nucleic acids, genomic DNA, mRNA, etc. The term “biological sample” encompasses a clinical sample, and also includes cells in culture, cell supernatants, tissue samples, organs, bone marrow, and the like.

As used herein, the term “isolated,” in the context of a nucleic acid, is meant to describe a nucleic acid that is in an environment different from that in which the nucleic acid naturally occurs, or that is in an environment different from that which the nucleic acid was found. As used herein, an “isolated” nucleic acid is one that is substantially free of the nucleic acids or other macromolecules with which it is associated in nature. By substantially free is meant at least 50%, preferably at least 70%, more preferably at least 80%, and even more preferably at least 90% free of the materials with which it is associated in nature. As used herein, an “isolated” nucleic acid also refers to recombinant nucleic acids, which, by virtue of origin or manipulation: (1) are not associated with all or a portion of a nucleic acid with which it is associated in nature, (2) are linked to a nucleic acid other than that to which it is linked in nature, or (3) does not occur in nature.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a catalytic nucleic acid” includes a plurality of such catalytic nucleic acids and reference to “the fluorescent energy transfer (FET) labeled nucleic acid substrate” includes reference to one or more such substrates and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Methods and compositions are provided for detecting a plurality of target nucleic acids in a sample. Also provided are systems and kits for practicing the subject methods.

Detection Methods

The present invention provides a rapid and procedurally flexible method of detecting and quantitatively measuring target nucleic acid sequences of interest in a sample. This method is unique in that it simultaneously employs target amplification and detection via catalytic nucleic activity in a single reaction vessel. Moreover, it is unique in that the amplification product is a single nucleic acid molecule containing sequences of the target and the catalytic nucleic acid molecule.

In some embodiments, a subject method for detecting the presence of a target nucleic acid in a sample comprises:
(a) contacting the sample, under conditions permitting primer-initiated nucleic acid amplification and catalytic nucleic acid activity, with:
(i) a first nucleic acid, where the first nucleic acid comprises a DNA zymogene which encodes, but which itself is the anti-sense sequence of, a catalytic nucleic acid molecule, and a first nucleic acid primer that initiates synthesis of a first, enzymatically inactive nucleic acid amplification product that comprises a nucleotide sequence that is complementary to the target; and
(ii) a second nucleic acid primer that hybridizes to the first amplification product and initiates synthesis of a second, enzymatically active nucleic acid amplification product, which comprises a nucleotide sequence of the target nucleic acid, and the catalytic nucleic acid molecule; and
(b) detecting the presence of catalytic nucleic acid activity, thereby detecting the presence of the target nucleic acid in the sample.

The present invention provides a method for detecting the presence of a target nucleic acid in a sample, the method generally involving:
(a) contacting the sample with:
(i) a first nucleic acid, wherein the first nucleic acid comprises a DNA zymogene and a first primer that initiates synthesis of a first, enzymatically inactive nucleic acid amplification product that comprises a nucleotide sequence that is complementary to the target nucleic acid;
(ii) a second primer nucleic acid that hybridizes to the first amplification product and initiates synthesis of a second, enzymatically active nucleic acid amplification product, which comprises a nucleotide sequence of the target nucleic acid, and the catalytic nucleic acid molecule; and
(iii) a third nucleic acid that comprises a nucleotide sequence that is complementary to the target nucleic acid, which nucleotide sequence at least partially overlaps with the nucleotide sequence of the first nucleic acid primer; and
(b) detecting the presence of catalytic nucleic acid activity, wherein the presence of catalytic nucleic acid activity indicates the presence of the target nucleic acid in the sample.

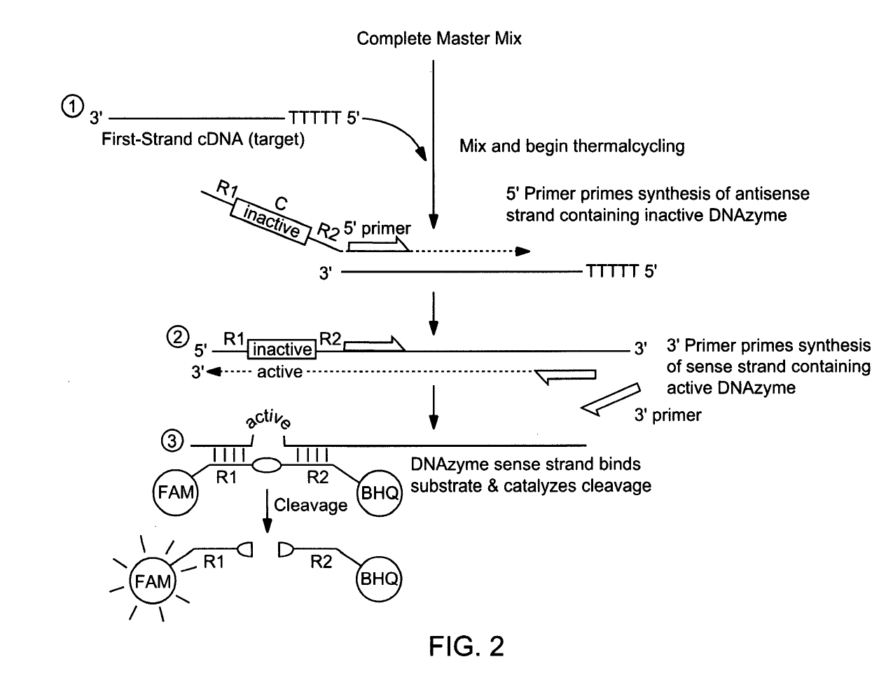

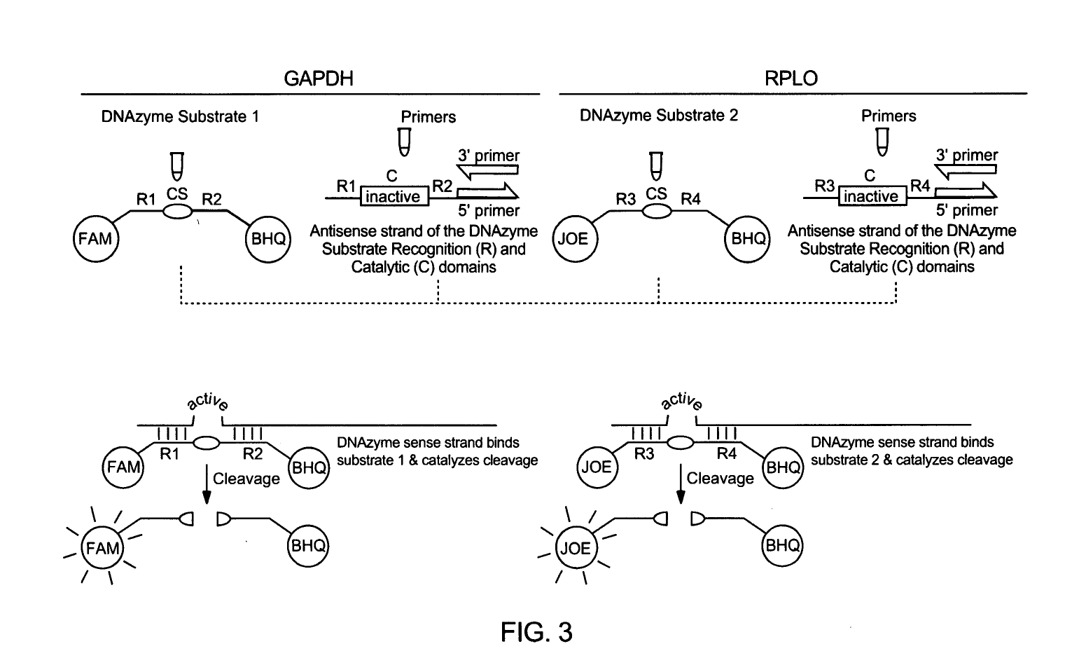

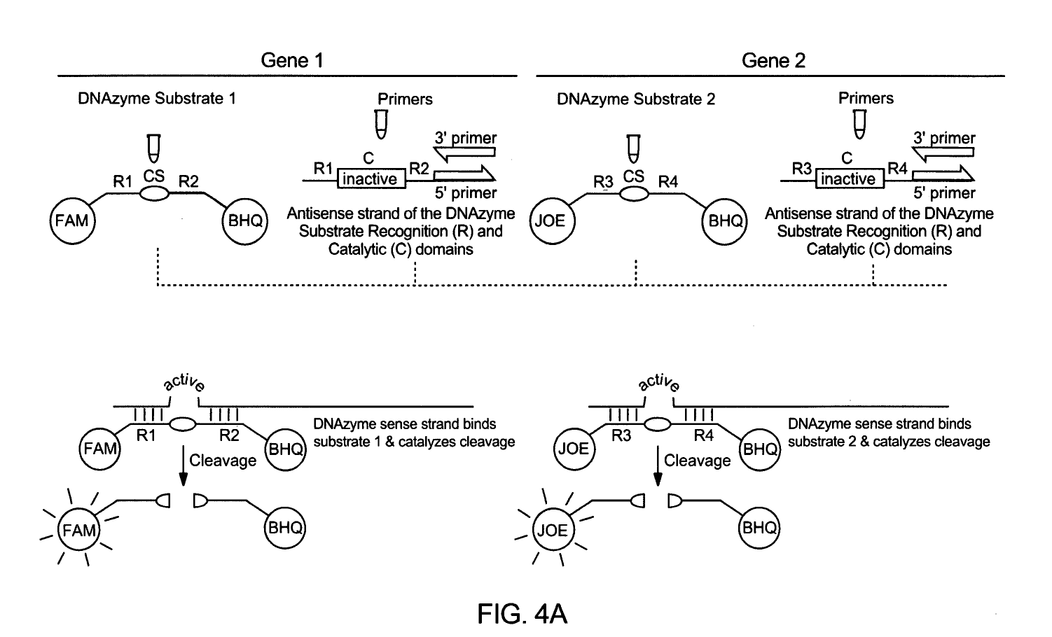

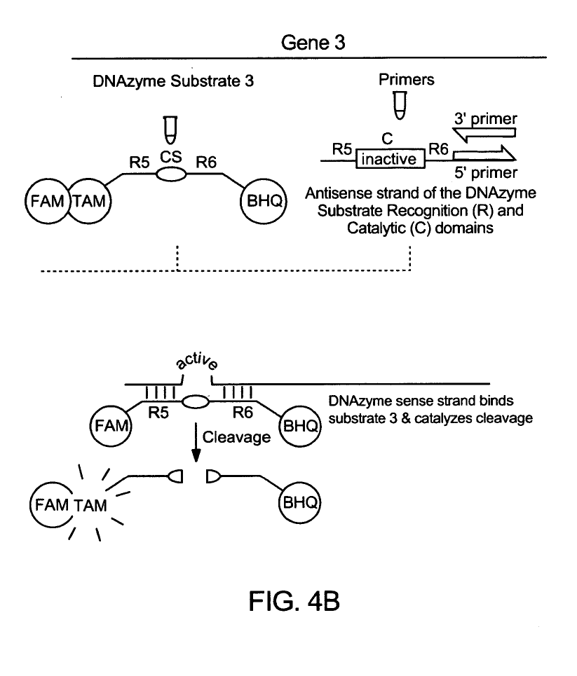

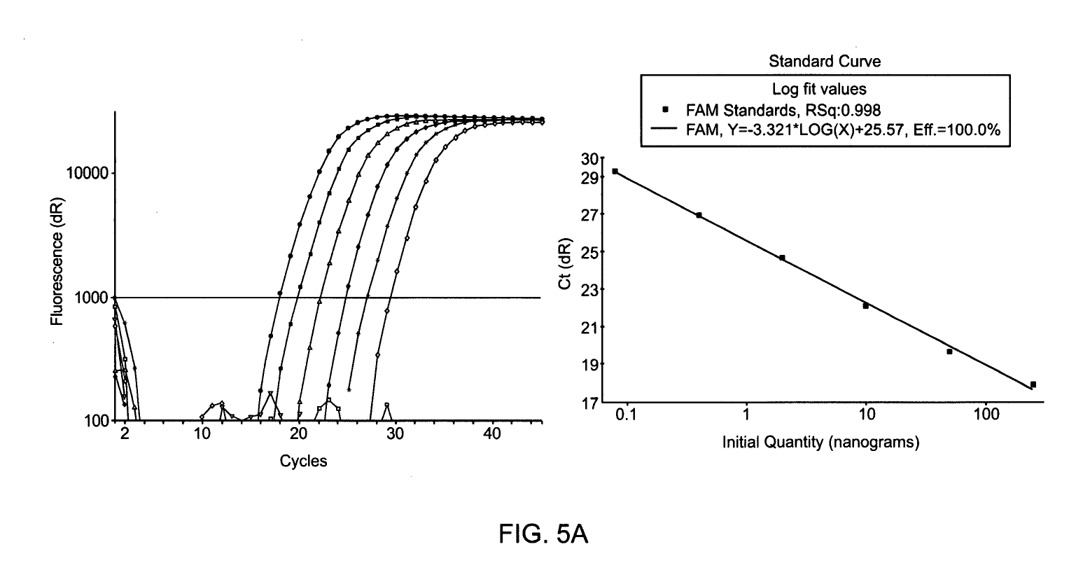

In one embodiment, the instant method further comprises the step of quantitatively determining the amount of catalytic nucleic acid activity in the sample resulting from step (a), and comparing the amount of activity so determined to a known standard, thereby quantitatively determining the amount of the target nucleic acid sequence. The known standard can be any standard or control used for quantitative determination. Examples of these standards include (i) known reaction kinetic information, as well as (ii) signal measurements obtained using samples containing no catalytic activity, or a pre-determined amount of catalytic activity. Non-limiting exemplary embodiments are depicted schematically in FIGS. 2, 3, and 4A and 4B. FIG. 2 depicts exemplary components of an amplification and detection reaction, where R1 and R2 are substrate recognition domains; C is a catalytic domain; BHQ is a Black Hole Quencher dye. DNA strands as follows: (1), target nucleic acid; (2), first, enzymatically inactive amplification product; and (3) second, enzymatically active amplification product. FIG. 3 is a schematic depiction of an exemplary duplex analysis, where two different target nucleic acids are detected in a single reaction mixture. FIGS. 4A and 4B present a schematic depiction of an exemplary triplex analysis, where three different target nucleic acids are detected in a single reaction mixture.

Zymogenes

The first nucleic acid (also referred to as a “zymogene/primer”) comprises, in order from 5′ to 3′, a DNA zymogene which encodes, but which itself is the anti-sense sequence of, a catalytic nucleic acid molecule, and a first nucleic acid primer that initiates synthesis of a first, enzymatically inactive nucleic acid amplification product that comprises a nucleotide sequence that is complementary to the target. The first nucleic acid primer is also referred to as the “5′ primer.”

The first nucleic acid primer initiates, or “primes,” synthesis of a first, enzymatically inactive nucleic acid amplification product that comprises a nucleotide sequence that is complementary to a nucleotide sequence in the target nucleic acid. The first nucleic acid primer has a length of from about 10 nucleotides to about 60 nucleotides, e.g., from about 10 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, or from about 50 nucleotides to about 60 nucleotides. The nucleotide sequence of the first primer is chosen based on the nucleotide sequence present within a target nucleic acid to be detected.

The zymogene domain of this first nucleic acid ranges in length from about 20 nucleotides to about 100 nucleotides, e.g., from about 20 nucleotides to about 25 nucleotides, from about 25 nucleotides to about 30 nucleotides, from about 30 nucleotides to about 40 nucleotides, from about 40 nucleotides to about 50 nucleotides, from about 50 nucleotides to about 60 nucleotides, from about 60 nucleotides to about 75 nucleotides, or from about 75 nucleotides to about 100 nucleotides. The nucleotide sequence of the zymogene encodes a catalytic nucleic acid, which catalytic nucleic acid comprises: a) a recognition nucleotide sequence that is complementary to a recognition nucleotide sequence of a corresponding substrate; and b) a catalytic domain which cleaves a catalytic substrate sequence in the substrate.

Representative nucleotide sequences of zymogenes include, but are not limited to, the following 5′ primer, which allows detection of RPP14 (GenBank accession number NM_{—}007042): 5′-TTGACGATACAGCTGCCACCAGGGCCTAGCTACAACGATCCAACTACCACAA GTCAGAAGCTTCCAAATCCTG-3′ (SEQ ID NO:1; zymogene sequence underlined) and 3′ primer: 5′-AAGTGGGCATTTGAACAAC-3′ (SEQ ID NO:2). See, e.g., U.S. Pat. No. 6,140,055. In some embodiments, the zymogene comprises a nucleotide sequence complementary to any one of SEQ ID NOs:3-17, as discussed below, which are catalytic nucleic acids.

The first nucleic acid primer hybridizes under stringent hybridization conditions to a complementary nucleotide sequence in the target nucleic acid. The first nucleic acid primer comprises a nucleotide sequence that is at least about 70%, at least about 75%, at least about 80%, at least about 85%, at least about 90%, at least about 95%, at least about 98%, at least about 99%, or 100% identical in nucleotide sequence to the complement of a nucleotide sequence of the same length in the target nucleic acid.

3′ Primer

The second nucleic acid primer hybridizes to the first, enzymatically inactive nucleic acid amplification product (also referred to as a “first, enzymatically inactive nucleic acid primer extension product”), and initiates synthesis of a second, enzymatically active nucleic acid amplification product (also referred to as a “second, enzymatically active nucleic acid primer extension product”) that comprises a nucleotide sequence found in the target nucleic acid; and an enzymatically active DNAzyme. Detection of the second, enzymatically active nucleic acid amplification product indicates the presence in the sample of the target nucleic acid.

In some embodiments, step (a) is carried out in the presence of a second nucleic acid primer, which second nucleic acid primer is “validated,” e.g., in a control amplification reaction in the absence of sample (e.g., in the absence of target nucleic acid), substantially no enzymatically active amplification product is generated. Thus, in some embodiments, the second nucleic acid primer in step (a) is one in which, in a reaction that includes a DNA polymerase and the first nucleic acid, but lacks sample (e.g., lacks a target nucleic acid), substantially no enzymatically active amplification product is generated (e.g., substantially no enzymatically active second nucleic acid amplification product or primer extension product, is generated). Such a second nucleic acid primer is referred to herein as a “validated 3′ primer” or a “validated second nucleic acid primer.” A feature of this validated 3′ primer is that it has been empirically determined to have the above functional properties. A reaction that proceeds in the absence of sample (e.g., in the absence of a target nucleic acid) is also referred to as a “no template control.” Substantially no enzymatically active nucleic acid amplification product is generated when the amount of enzymatically active nucleic acid amplification product is at or below the detection limit.

Alternatively, a validated second nucleic acid primer differs in base sequence from a non-validated second nucleic acid primer, such that one, two, three, or more mismatches to the target sequence are introduced.

Driver Primer

In some embodiments, step (a) is carried out in the presence of a third nucleic acid that includes at least a portion of the nucleotide sequence of the first nucleic acid primer (and not the zymogene). The third nucleic acid is also referred to herein as “the driver primer.” In these embodiments, a subject method for detecting the presence of a target nucleic acid in a sample involves: (a) contacting the sample with: (i) a first nucleic acid, where the first nucleic acid includes a DNA zymogene which encodes, but which itself is the anti-sense sequence of, a catalytic nucleic acid; and a first primer that initiates synthesis of a first, enzymatically inactive nucleic acid amplification product that comprises a nucleotide sequence that is complementary to the target nucleic acid; (ii) a second primer nucleic acid that hybridizes to the first amplification product and initiates synthesis of a second, enzymatically active nucleic acid amplification product, which comprises a nucleotide sequence of the target nucleic acid, and the catalytic nucleic acid molecule; and (iii) a third nucleic acid, where the third nucleic acid includes at least a portion of the nucleotide sequence of the first nucleic acid primer, and does not include the zymogene; and (b) detecting the presence of catalytic nucleic acid activity, where the presence of catalytic nucleic acid activity indicates the presence of the target nucleic acid in the sample.

In these embodiments, the third nucleic acid is present in the sample in a molar ratio of at least about 1:1 with the first nucleic acid. The third nucleic acid is present in the sample in a molar ratio of from about 1:1 to about 100:1 with the first nucleic acid, e.g., the third nucleic acid is present in the sample in a molar ratio of from about 1:1 to about 1.5:1, from about 1.5:1 to about 2:1, from about 2:1 to about 2.5:1, from about 3:1 to about 4:1, from about 4:1 to about 5:1, from about 5:1 to about 10:1, from about 10:1 to about 20:1, from about 20:1 to about 25:1, from about 25:1 to about 50:1, from about 50:1 to about 75:1, or from about 75:1 to about 100:1.

The third nucleic acid has a length of from about 10 nucleotides to about 60 nucleotides, e.g., from about 10 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, or from about 50 nucleotides to about 60 nucleotides. The third nucleic acid includes from about 5 nucleotides up to the entire nucleotide sequence of the first nucleic acid primer, and may include additional nucleotide sequences that are complementary to and/or hybridize to the target nucleic acid.

The third nucleic acid is present in the reaction of step (a) in a concentration of from about 5 nM to about 300 nM, e.g., from about 5 nM to about 10 nM, from about 10 nM to about 25 nM, from about 25 nM to about 50 nM, from about 50 nM to about 75 nM, from about 75 nM to about 100 nM, from about 100 nM to about 150 nM, from about 150 nM to about 200 nM, from about 200 nM to about 250 nM, or from about 250 nM to about 300 nM.

The efficiency of the amplification reaction in the presence of the third nucleic acid is increased by about 10% to about 110%, e.g., from about 10% to about 25%, from about 25% to about 50%, or from about 50% to about 110%, compared to the efficiency of the amplification reaction in the absence of the third DNA molecule, as determined using the protocol described in the Experimental section, below. In the presence of the third DNA molecule, the amplification reaction proceeds with an efficiency of from about 80% to about 110%, e.g., from about 80% to about 90%, from about 90% to about 95%, from about 95% to about 98%, from about 98% to about 100%, or from about 100% to about 110%, where efficiency is calculated from the slope of the standard curve of cycle threshold (Ct) versus log₁₀ amount of starting material. Specifically, efficiency is defined as: E=(10\hat{ }(−1/slope))−1, where the slope is the slope of the standard curve generated as previously described, namely from the plot of Ct versus the log of the amount of starting material.

In some embodiments, the third nucleic acid is blocked, e.g., is incapable of priming synthesis, and does not prime synthesis of a nucleic acid complementary to the target nucleic acid. The third nucleic acid is blocked in any of a number of ways, e.g., by removal of the 3′ OH of the 3′ nucleotide, by modification of the 3′ OH of the 3′ nucleotide, or by some other type of 3′ base modification. For example, in some embodiments, the third nucleic acid includes at its 3′ end a dideoxyribonucleotide. In other embodiments, the third nucleic acid includes at its 3′ end a nucleotide, where the 3′ OH of the nucleotide is phosphorylated. In other embodiments, the third nucleic acid comprises a 3′-O-methyl group. In other embodiments, the third nucleic acid comprises a 3′ inverted base. Other types of 3′ base modifications that block chain elongation and/or that block priming are known to those skilled in the art, and can be used in a subject composition and/or method.

Reaction Conditions

Conditions that permit primer-initiated nucleic acid amplification and catalytic nucleic acid activity are well known to those skilled in the art, and include the presence of a DNA polymerase; deoxynucleotide triphosphates; and magnesium ions. Suitable reaction conditions are well known to those skilled in the art of nucleic acid amplification. Exemplary, non-limiting reaction conditions are described in the Examples. The DNA polymerase is generally one that has high affinity for binding at the 3′-end of an oligonucleotide hybridized to a nucleic acid strand. The DNA polymerase is generally one that has little or no 5′→3′ exonuclease activity so as to minimize degradation of primer, termination or primer extension polynucleotides. The DNA polymerase is generally one that has little to no proofreading activity. In many embodiments, the DNA polymerase is thermostable, e.g., is catalytically active at temperatures in excess of about 75° C. DNA polymerases that are suitable for use in a subject method include, but are not limited to, DNA polymerases discussed in U.S. Pat. Nos. 5,648,211 and 5,744,312, which include exo⁻ Vent (New England Biolabs), exo⁻ Deep Vent (New England Biolabs), Bst (BioRad), exo⁻ Pfu (Stratagene), Bca (Panvera), sequencing grade Taq (Promega); thermostable DNA polymerases from Thermoanaerobacter thermohydrosulfuricus; and the like. In some embodiments, the reaction mixture includes an RNAse H.

Magnesium ions are typically present in the reaction mix in a concentration of from about 1 mM to about 100 mM, e.g., from about 1 mM to about 3 mM, from about 3 mM to about 5 mM, from about 5 mM to about 10 mM, from about 10 mM to about 25 mM, from about 25 mM to about 50 mM, from about 50 mM to about 75 mM, or from about 75 mM to about 100 mM.

Usually the reaction mixture will comprise four different types of dNTPs corresponding to the four naturally occurring bases are present, i.e. dATP, dTTP, dCTP and dGTP. In the subject methods, each dNTP will typically be present at a final concentration in the reaction, ranging from about 10 μM to 5000 μM, e.g., from about 10 μM to about 50 μM, from about 50 μM to about 100 μM, from about 100 μM to about 200 μM, from about 200 μM to about 500 μM, from about 500 μM to about 1000 μM, from about 1000 μM to about 2000 μM, from about 2000 μM to about 3000 μM, from about 3000 μM to about 4000 μM, or from about 4000 μM to about 5000 μM. In many embodiments, each dNTP will be present at a final concentration in the reaction of from about 20 μM to 1000 μM, from about 100 μM to about 200 μM, or from about 50 μM to about 200 μM.

The reaction mixture prepared in the first step of the subject methods further includes an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulfate, and the like may be employed. The divalent cation may be magnesium, manganese, zinc and the like, where the cation will typically be magnesium. Any convenient source of magnesium cation may be employed, including MgCl₂, Mg-acetate, and the like. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, e.g., pH 7.3 at 72° C. Other agents which may be present in the buffer medium include chelating agents, such as EDTA, EGTA and the like.

Each primer nucleic acid is present in the reaction mixture at a concentration of from about 50 nM to about 900 nM, e.g., the 3′ primer and the 5′ primer nucleic acid are each independently present at a concentration of from about 50 nM to about 75 nM, from about 75 nM to about 100 nM, from about 100 nM to about 150 nM, from about 150 nM to about 200 nM, from about 200 nM to about 250 nM, from about 250 nM to about 300 nM, from about 300 nM to about 400 nM, from about 400 nM to about 500 nM, from about 500 nM to about 600 nM, from about 600 nM to about 700 nM, from about 700 nM to about 800 nM, or from about 800 nM to about 900 nM.

Catalytic Nucleic Acids

As discussed above, the subject method provides for generation of a second, enzymatically active nucleic acid amplification product (“catalytic nucleic acid”). The catalytic nucleic acid encoded by the zymogene portion of the first nucleic acid recognizes and cleaves a sequence present in a substrate. In addition, the catalytic nucleic acid comprises a target nucleotide sequence, e.g., a nucleotide sequence found in a target nucleic acid. The enzymatically active portion of the catalytic nucleic acid encoded by the zymogene is a ribozyme or a DNAzyme. In one embodiment, the catalytic nucleic acid molecule is a ribozyme. In many embodiments, the catalytic nucleic acid molecule is a DNAzyme.

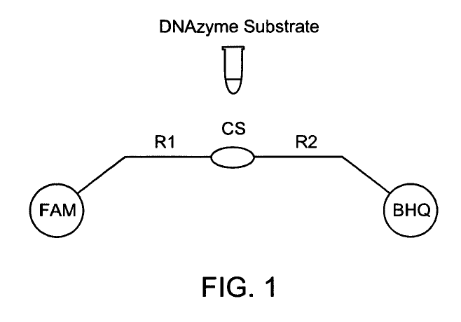

In some embodiments, as depicted in FIG. 1, a catalytic nucleic acid comprises first recognition domain, R1; a catalytic domain, C; and a second recognition domain, R2. Each of R1, C, and R2 independently has a length of from about 10 nucleotides to about 60 nucleotides, e.g., from about 10 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, or from about 50 nucleotides to about 60 nucleotides. The recognition domains hybridize, e.g., under stringent hybridization conditions, to complementary nucleotide sequences in a substrate, as described in more detail below.

Catalytic nucleic acids comprise a catalytic domain. Catalytic nucleic acids are known in the art, and any catalytic domain can be used in the instant invention. In some embodiments, the catalytic nucleic acid comprises a Mg²⁺-dependent catalytic domain. A non-limiting example of a Mg²⁺-dependent catalytic domains is the 15-nucleotide sequence 5′-GGCTAGCTACAACGA-3′ (SEQ ID NO:3). In some embodiments, the catalytic nucleic acid comprises a Zn²⁺-dependent catalytic domain. In some embodiments, the catalytic nucleic acid is a DNAzyme. In some embodiments, the DNAzyme comprises a catalytic motif comprising the nucleotide sequence 5′-GGCTAGCTACAACGA-3′ (SEQ ID NO:3). In some embodiments, the DNAzyme is a 10-23 DNAzyme comprising a catalytic motif having the nucleotide sequence set forth in SEQ ID NO:3. See, e.g., FIG. 2 of Santoro and Joyce (1997) Proc. Natl. Acad. Sci. USA 94:4262-4266. In other embodiments, the DNAzyme comprises a catalytic motif comprising the nucleotide sequence 5′-TCCGAGCCGGACGA-3′ (SEQ ID NO:4), 5′-CCGAGCCGGACGA-3′ (SEQ ID NO:5), or 5′-CATATACT CCGAGCCGGACGACACGTCGC-3′ (SEQ ID NO:6). In some embodiments, the DNAzyme is an 8-17 DNAzyme that comprises a nucleotide sequence as set forth in SEQ ID NO:2, 3, or 4. See, e.g., See, e.g., FIG. 2 of Santoro and Joyce (1997) supra; FIG. 1 of Peracchi (2000) J. Biol. Chem. 275:11693-11697; and the variants listed in FIG. 2 of Peracchi (2000), supra. In other embodiments, the DNAzyme comprises a catalytic motif comprising the nucleotide sequence 5′-GTCAGCGACACGAA-3′ (SEQ ID NO:7) or 5′-GCTGTTGATCTGTCAGCGACACGAAATGGTGAT-3′ (SEQ ID NO:8). In some embodiments, the DNAzyme is an Mg5 DNAzyme that comprises a nucleotide sequence as set forth in SEQ ID NO:5 or 6. See, e.g., FIG. 4 of Peracchi (2000), supra. In other embodiments, the DNAzyme comprises a catalytic motif comprising the nucleotide sequence 5′-GGAGAACGCGAGGCAAGGCTGGGAGAAATGTGGATCACGATT-3′ (SEQ ID NO:9) or 5′-GGAGAACGCGAGGCAAGGCTGGGAGAAATGTGGATCACGATTGTCTGAATC GGTCTGTATC-3′ (SEQ ID NO:10). See, e.g., Chinnapen and Sen (2004) Proc. Natl. Acad. Sci. USA 101:65-69. In other embodiments, the DNAzyme comprises a catalytic motif comprising the nucleotide sequence 5′-ACGTGGAGGTGGGCTC-3′ (SEQ ID NO:11). In some embodiments, the DNAzyme is a 7Q10 DNAzyme that comprises the nucleotide sequence set forth in SEQ ID NO:9. See, e.g., FIG. 1 of Ricca et al. (2003) J. Mol. Biol. 330:1015-1025. In other embodiments, the DNAzyme comprises a catalytic motif comprising the nucleotide sequence 5′-CATCTCTTCTCCGAGCCGGTCGAAATAGTGAGT-3′ (SEQ ID NO:12), where the catalytic activity is Zn²⁺ dependent. In other embodiments, the DNAzyme comprises a catalytic motif comprising the nucleotide sequence 5′-AGCGATCCGGAACGGCACCCATGT-3′ (SEQ ID NO:13). In other embodiments, the DNAzyme comprises a catalytic motif comprising the nucleotide sequence 5′-GGCTAGCTACAACGA-3′ (SEQ ID NO:14). In other embodiments, the DNAzyme comprises a catalytic motif comprising the nucleotide sequence 5′-AACGGGGCTGTGCGGCTAGGAAGTA-3′ (SEQ ID NO:15). In other embodiments, the DNAzyme comprises a catalytic motif comprising the nucleotide sequence 5′-GAAGTAGCGCCGCCG-3′ (SEQ ID NO:16). In other embodiments, the DNAzyme comprises a catalytic motif comprising the nucleotide sequence 5′-CCGAGCCGGTCGAA-3′ (SEQ ID NO:17). In other embodiments, the DNAzyme comprises a catalytic domain comprising a nucleotide sequence as depicted in U.S. Pat. No. 6,706,474.

The catalytic nucleic acid activity measured in the instant methods can be any activity which can occur (and, optionally, be measured) simultaneously and in the same milieu with a nucleic acid amplification reaction. The catalytic nucleic acid activity can comprise, for example, the modification of a detectable chemical substrate, which modification is selected from the group consisting of phosphodiester bond formation and cleavage, nucleic acid ligation and cleavage, porphyrin metallation, and formation of carbon-carbon, ester and amide bonds. In one embodiment, the detectable chemical substrate modification is cleavage of a fluorescently labeled nucleic acid molecule, and in many embodiments a FET-labeled substrate, as described below in greater detail.

In certain embodiments, the reporter substrate is cleaved, and measuring this cleavage is a means of measuring the catalytic activity. For example, the presence of the cleaved substrate can be monitored by phosphorimaging following gel electrophoresis provided the reporter substrate is radiolabelled. The presence of cleaved substrate can also be monitored by changes in fluorescence resulting from the separation of fluoro/quencher dye molecules incorporated into opposite sides of the cleavage site within the substrate. Such systems provide the opportunity for a homogeneous assay which can be monitored in real time.

FET-Labeled Oligonucleotide Substrate

A feature of certain embodiments of the subject invention is that it employs FET-labeled oligonucleotide substrates to detect the presence of the catalytic activity produced for each target nucleic acid according to the subject invention. FET occurs when a suitable fluorescent energy donor and an energy acceptor moiety are in close proximity to one another. The excitation energy absorbed by the donor is transferred to the acceptor which can then further dissipate this energy either by fluorescent emission if a fluorophore, or by non-fluorescent means if a quencher. A donor-acceptor pair comprises two fluorophores having overlapping spectra, where the donor emission overlaps the acceptor absorption, so that there is energy transfer from the excited fluorophore to the other member of the pair. It is not essential that the excited fluorophore actually fluoresce, it being sufficient that the excited fluorophore be able to efficiently absorb the excitation energy and efficiently transfer it to the emitting fluorophore.

As such, the FET-labeled oligonucleotides employed in the subject methods are nucleic acid detectors that include a fluorophore domain where the fluorescent energy donor, i.e., donor, is positioned and an acceptor domain where the fluorescent energy acceptor, i.e., acceptor, is positioned. As mentioned above, the donor domain includes the donor fluorophore. The donor fluorophore may be positioned anywhere in the nucleic acid detector, but is typically present at the 5′ terminus of the detector.

The acceptor domain includes the fluorescence energy acceptor. The acceptor may be positioned anywhere in the acceptor domain, but is typically present at the 3′ terminus of the nucleic acid detector.

The nucleic acid detector comprises: a) a first recognition nucleotide sequence that is complementary to the first recognition nucleotide sequence on the second, enzymatically active nucleic acid amplification product; b) a substrate for the catalytic domain of the catalytic nucleic acid; and c) a second recognition nucleotide sequence that is complementary to the second recognition nucleotide sequence on the second, enzymatically active nucleic acid amplification product.

Thus, as depicted in FIG. 2, in many embodiments, a FET-labeled substrate comprises a first recognition domain, R1; a catalytic substrate domain, CS; and a second recognition domain, R2. Each of R1, C, and R2 independently has a length of from about 10 nucleotides to about 60 nucleotides, e.g., from about 10 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, or from about 50 nucleotides to about 60 nucleotides. The recognition domains R1 and R2 hybridize to complementary nucleotide sequences R1 and R2 in the catalytic nucleic acid (e.g., a DNAzyme). The catalytic domain of the catalytic nucleic acid recognizes and cleaves a site in the catalytic substrate domain.

The overall length of the FET-labeled oligonucleotide substrate, which includes all three domains mentioned above, typically ranges from about 10 nucleotides to about 60 nucleotides, e.g., from about 15 nucleotides to about 25 nucleotides, from about 25 nucleotides to about 30 nucleotides, from about 30 nucleotides to about 40 nucleotides, from about 40 nucleotides to about 50 nucleotides, or from about 50 nucleotides to about 60 nucleotides.

The donor fluorophore of the subject FET-labeled substrate is typically one that is excited efficiently by a single light source of narrow bandwidth, particularly a laser source. The emitting or accepting fluorophores are selected to be able to receive the energy from the donor fluorophore and emit light. Usually the donor fluorophores will absorb in the range of about 350-800 nm, e.g., in the range of about 350-600 nm, or 500-750 nm. The transfer of the optical excitation from the donor to the acceptor depends on the distance between the two fluorophores. Thus, the distance must be chosen to provide efficient energy transfer from the donor to the acceptor. The distance between the donor and acceptor moieties on the FET-labeled oligonucleotides employed in the subject invention, at least in certain configurations (such as upon intramolecular association) typically ranges from about 10 to about 100 angstroms.

Suitable fluorophores for FET include, but are not limited to, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethylrhodamine-, methyl ester), TMRE (tetramethylrhodamine, ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; 5-(2′-aminoethyl)aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes; and the like. Fluorophores of interest are further described in WO 01/42505 and WO 01/86001.

Quenchers that can be used in the methods provided herein include, but are not limited to, diarylrhodamine derivatives, such as the QSY 7, QSY 9, and QSY 21 dyes available from Molecular Probes; dabcyl and dabcyl succinimidyl ester; dabsyl and dabsyl succinimidyl ester; QSY 35 acetic acid succinimidyl ester; QSY 35 iodoacetamide and aliphatic methylamine; a Black Hole Quencher (BHQ), e.g., BHQ-0, BHQ-1, BHQ-2, etc.; napthalate; and Cy5Q and Cy7Q from Amersham Biosciences.

In certain embodiments, the acceptor moiety of at least one of the FET-labeled substrates is a quencher molecule, e.g., a molecule that absorbs transferred energy but does not emit fluorescence, e.g., a dark quencher. In many embodiments, the dark quencher has maximum absorbance of between about 400 nm and about 700 nm, e.g., between about 400 nm and about 500 nm, between about 500 nm and about 600 nm, or between about 600 nm and about 700 nm.

The fluorescence donor will be paired with a suitable fluorescence acceptor. For example, BHQ-0 has a λmax of 495 nm (range=430-520 nm); BHQ-1 has a λmax of 534 nm (range 480-580 nm); and BHQ-2 has a λmax of 579 nm (range 550-650 nm). The quencher will be paired with an appropriate fluorescence donor (e.g., TAM, TAMRA, JOE, etc.; or a FRET pair such as JOE/ROX, FAM/TAM, JOE/TAM, etc.). Selection of an appropriate fluorescence donor and fluorescence acceptor is well within the skill level of those of ordinary skill in the art.

In certain embodiments, the dark quencher comprises a substituted 4-(phenyldiazenyl)phenylamine structure, often comprising at least two residues selected from aryl, substituted aryl, heteroaryl, substituted heteroaryl and combination thereof, wherein at least two of said residues are covalently linked via an exocyclic diazo bond.

In certain embodiments, the dark quencher is described by the following formula: 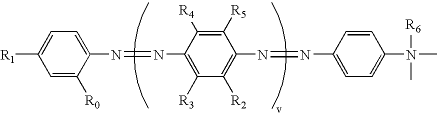
wherein:
R₀, R₁, R₂, R₃, R₄, R₅ are independently: —H, halogen, —O(CH₂)ₙCH₃, —(CH₂)ₙCH₃, —NO₂, SO₃, —N[(CH₂)ₙCH₃]₂ wherein n=0 to 5 or —CN; R₆ is —H or —(CH₂)ₙCH₃ where n=0 to 5; and v is a number from 0 to 10.

Dark quenchers of interest are further described in WO 01/42505 and WO 01/86001.

The FET-labeled oligonucleotide substrates may be structured in a variety of different ways, so long as it includes the above-described donor, acceptor and nucleic acid binding domains.

Methods for monitoring changes in fluorescence are well known in the art. Such methods include, by way of example, visual observation and monitoring with a spectrofluorometer.

Target Nucleic Acids

The target sequence detected or quantitated in the instant methods can be any nucleic acid sequence. In one embodiment, the target nucleic acid sequence is a DNA molecule. In another embodiment, the target nucleic acid sequence is an RNA molecule, and step (a) further comprises the step of first reverse transcribing the target RNA sequence to a complementary DNA prior to contacting the sample with the 3′ primer and zymogene. In some embodiments, the target is the coding strand of a nucleic acid. In other embodiments, the target nucleic acid is the non-coding strand of a nucleic acid.

The target nucleic acid sequence can be from any organism, and the sample can be any composition containing, or suspected to contain, nucleic acid molecules. In one embodiment, the target nucleic acid is found in a cell or organism of any of the six kingdoms, e.g., Bacteria (e.g., Eubacteria); Archaebacteria; Protista; Fungi; Plantae; and Animalia. Target nucleic acids include nucleic acids from cells or organisms that include, but are not limited to, a protozoan, a plant, a fungus, an algae, a yeast, a reptile, an amphibian, a mammal, a marine microorganism, a marine invertebrate, an arthropod, an isopod, an insect, an arachnid, an archaebacterium, and a eubacterium. Mammalian sources of target nucleic acids include, but are not limited to, primates, felines, canines, ungulates, equines, bovines, ovines, etc.

The target nucleic acid may be derived from a variety of different sources, depending on the application for which the subject method is being performed, where such sources include organisms that comprise nucleic acids, i.e. viruses; prokaryotes, e.g. bacteria, archaea and cyanobacteria; and eukaryotes, e.g. members of the kingdom protista, such as flagellates, amoebas and their relatives, amoeboid parasites, ciliates and the like; members of the kingdom fungi, such as slime molds, acellular slime molds, cellular slime molds, water molds, true molds, conjugating fungi, sac fungi, club fungi, imperfect fungi and the like; plants, such as algae, mosses, liverworts, hornworts, club mosses, horsetails, ferns, gymnosperms and flowering plants, both monocots and dicots; and animals, including sponges, members of the phylum cnidaria, e.g. jelly fish, corals and the like, combjellies, worms, rotifers, roundworms, annelids, molluscs, arthropods, echinoderms, acorn worms, and vertebrates, including reptiles, fishes, birds, snakes, and mammals, e.g. rodents, primates, including humans, and the like. The target nucleic acid may be used directly from its naturally occurring source and/or preprocessed in a number of different ways, as is known in the art. In some embodiments, the target nucleic acid is from a synthetic source.

In some embodiments, target nucleic acid will be from a tissue taken from an organism; from a particular cell or group of cells isolated from an organism; etc. For example, where the organism is a plant, the nucleic acid will in some embodiments be from the xylem, the phloem, the cambium layer, leaves, roots, etc. Where the organism is an animal, the nucleic acid will in some embodiments be isolated from a particular tissue (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.). In another embodiment, the target is in a sample obtained from a source such as water or soil. In a further embodiment, the target is from a sample containing bacteria, viruses or mycoplasma.

In certain embodiments, the target nucleic acid is from a human cell, organ, tissue, or other biological sample containing nucleic acids. The instant methods can be used for a variety of purposes including, for example, diagnostic, public health and forensic.

In some embodiments, the target nucleic acid is present in a biological sample, e.g., a nucleic acid-containing sample obtained from an individual. Suitable biological samples include any that contain nucleic acids. Suitable biological samples include, but are not limited to, blood; solid tissue samples such as a biopsy specimen; bone marrow; cells obtained from the individual and cultured in vitro, and cells derived therefrom and the progeny thereof; sputum; vaginal swabs; oral swabs; bronchoalveolar lavage; and the like. Suitable biological samples also include samples that have been manipulated in any way after their procurement, such as by treatment with reagents; washed; or enrichment for certain cell populations, such as CD4⁺ T lymphocytes, glial cells, macrophages, tumor cells, peripheral blood mononuclear cells (PBMC), and the like; or enrichment for particular macromolecules, e.g., nucleic acids, genomic DNA, mRNA, etc.

In another embodiment, the sample being tested for the presence or amount of target nucleic acid molecule is a sample taken for public health purposes. Examples of such samples include water, food, and soil, possibly containing harmful pathogens such as bacteria, viruses, protozoa, helminths, and mycoplasma.

In a further embodiment, the sample being tested for the presence or amount of target nucleic acid molecules is a forensic sample. Examples of such samples include bodily fluids, tissues and cells, which can be obtained from any source such as a crime scene.

In some embodiments, nucleic acids containing a target nucleic acid, or nucleic acids to be tested for the presence of a target nucleic acid, are isolated from the cell, organism, or biological sample containing the target nucleic acid. In some embodiments, nucleic acids containing a target nucleic acid, or nucleic acids to be tested for the presence of a target nucleic acid are purified, e.g., the nucleic acids are at least 80%, at least 95%, at least 97%, or at least 99% pure. Isolation and/or purification of nucleic acids is accomplished using well-established methods.

Detection of Multiple Target Nucleic Acids

This invention also provides a method of simultaneously detecting the presence of a plurality of target nucleic acid sequences in a sample. The method generally involves: (a) contacting the sample with: (i) a plurality of zymogenes, where for each target being detected, there exists at least one zymogene which encodes, but which itself is the anti-sense sequence of, a catalytic nucleic acid molecule having distinctly measurable catalytic activity, where each zymogene comprises a different first nucleic acid primer that initiates synthesis of a different first, enzymatically inactive nucleic acid amplification product that comprises a nucleotide sequence that is complementary to a target nucleic acid; and (ii) a plurality of second nucleic acid primers, where for each target being detected, there exists at least one second nucleic acid primer that hybridizes to one of the first amplification products and initiates synthesis of a second, enzymatically active nucleic acid amplification product, which second amplification product comprises a nucleotide sequence of one of the target nucleic acids, and one of the catalytic nucleic acids; and (b) detecting the presence of each of the catalytic nucleic acid activities by employing a separate FET-labeled substrate for each of the catalytic activities, wherein at least one of the FET-labeled substrates include a dark quencher. Detection of catalytic activity associated with a particular FET-labeled substrate indicates the presence of the corresponding target nucleic acid sequence in the sample.

In one embodiment, the method of simultaneously detecting the presence of a plurality of targets further comprising the step of quantitatively determining the amount of each catalytic nucleic acid activity in the sample resulting from step (a), and comparing the amount of each activity so determined to a known standard, thereby quantitatively determining the amount of each target nucleic acid sequence.

In some embodiments, step (a) further comprises contacting the sample with a plurality of third nucleic acids (“5′ driver primers”), each of which comprises a nucleotide sequence that is complementary to a target nucleic acid, which nucleotide sequence at least partially overlaps with the nucleotide sequence one of the first nucleic acid primers. In some embodiments, step (a) is carried out in the presence of a plurality of third nucleic acids, where, for each first primer nucleic acid present in the plurality of zymogenes, a third nucleic acid is present, where each of the third nucleic acids comprise a nucleotide sequence that at least partially overlaps with a corresponding first nucleic acid primer. In some embodiments, one or more of the third nucleic acids is present in the sample at a molar ratio of at least about 1:1 with the corresponding zymogene. In some embodiments, one or more of the third nucleic acids is present in the sample at a molar ratio of at least about 1.5:1 with the corresponding zymogene. In some embodiments, one or more of the third nucleic acids is present in the sample at a molar ratio of at least about 2:1 with the corresponding zymogene. In some embodiments, one or more of the third nucleic acids is present in the sample at a molar ratio of at least about 5:1, about 10:1, or about 20:1, or higher, with the corresponding zymogene.

In some embodiments, at least one of the third nucleic acids comprises a 3′ OH modification such that the third nucleic acid does not initiate synthesis of a nucleic acid amplification product. In other embodiments, each of the third nucleic acid comprise a 3′ OH modification such that the third nucleic acid does not initiate synthesis of a nucleic acid amplification product.

In some embodiments, at least one of the second nucleic acid primers does not yield an enzymatically active nucleic acid amplification product in a control sample lacking a target nucleic acid, e.g., the reaction of step (a) is carried out in the presence of a plurality of 3′ primers (second nucleic acid primers), where at least one of the 3′ primers is a validated 3′ primer. In some embodiments, the reaction of step (a) is carried out in the presence of a plurality of validated 3′ primers, where, for each first nucleic acid amplification product, there is a corresponding second nucleic acid comprising a second nucleic acid primer, and where each of the second nucleic acid primers is validated.

In some embodiments, step (a) is carried out in the presence of both at least one 5′ driver primer and at least one 3′ validated primer.

In these embodiments, the sample comprises multiple (e.g., at least two) different target nucleic acids. The sample comprises from two different target nucleic acids to 20 or more different target nucleic acids, e.g., two different target nucleic acids, three different target nucleic acids, four different target nucleic acids, five different target nucleic acids, six different target nucleic acids, seven different target nucleic acids, eight different target nucleic acids, nine different target nucleic acids, ten different target nucleic acids, more than ten different target nucleic acids, or more than 20 different target nucleic acids.

Multiple target nucleic acids include, e.g., samples from two or more different mammalian subjects (e.g., two or more different crime suspects; etc.); samples from two or more different plant species of the same genus; samples from two or more different animal species of the same genus; two or more different bacterial species of the same genus; two or more bacterial strains (e.g., two or more bacterial strains of the same species); two or more different archaebacteria of the same genus; and the like.

Further examples of multiple targets which can be simultaneously detected by the instant methods are disclosed in, e.g., WO 96/32500.

Compositions

The present invention provides sets of FET-labeled oligonucleotide substrates, and compositions comprising the FET-labeled oligonucleotide substrate sets. The present invention further provides set of zymogenes, and compositions comprising the zymogene sets. Compositions will in some embodiments further include one or more of: a buffer; a salt; a pH adjusting agent; a detergent (e.g., a non-ionic detergent); a nuclease inhibitor; a preservative; and the like. Each FET-labeled oligonucleotide substrate in the set is present in the composition in an amount sufficient to carry out at least one detection reaction as described herein. Each zymogene in the set is present in the composition in an amount sufficient to carry out at least one detection reaction as described herein.

In some embodiments, each FET-labeled oligonucleotide substrate of the set is present in the composition at a concentration of from about 500 nM to about 10,000 nM, e.g., from about 500 nM to about 750 nM, from about 750 nM to about 1000 nM, from about 1000 nM to about 1500 nM, from about 1500 nM to about 2000 nM, from about 2000 nM to about 2500 nM, from about 2500 nM to about 3000 nM, from about 3000 nM to about 4000 nM, from about 4000 nM to about 5000 nM, from about 5000 nM to about 6000 nM, from about 6000 nM to about 7000 nM, from about 7000 nM to about 8000 nM, or from about 8000 nM to about 10,000 nM. In some embodiments, each FET-labeled oligonucleotide substrate of the set is present in the composition at a concentration greater than 10 μM, e.g., from about 10 μM to about 100 μM, or from about 100 μM to about 1 mM.

In some embodiments, each zymogene of the zymogene set is present in the composition at a concentration of from about 500 nM to about 10,000 nM, e.g., from about 500 nM to about 750 nM, from about 750 nM to about 1000 nM, from about 1000 nM to about 1500 nM, from about 1500 nM to about 2000 nM, from about 2000 nM to about 2500 nM, from about 2500 nM to about 3000 nM, from about 3000 nM to about 4000 nM, from about 4000 nM to about 5000 nM, from about 5000 nM to about 6000 nM, from about 6000 nM to about 7000 nM, from about 7000 nM to about 8000 nM, or from about 8000 nM to about 10,000 nM. In some embodiments, each zymogene of the set is present in the composition at a concentration greater than 10 μM, e.g., from about 10 μM to about 100 μM, or from about 100 μM to about 1 mM.

Sets of FET-Labeled Oligonucleotide Substrates

A subject method involving detection of multiple target nucleic acid employs a plurality of zymogenes; and a plurality of FET-labeled substrates, e.g., a different FET-labeled substrate for each of the catalytic activities. The present invention further provides sets of FET-labeled substrates for use in the subject methods; and compositions comprising the FET-labeled substrates.

A subject set of FET-labeled substrates comprises a collection of from about two to about 100, or more, different FET-labeled substrates. For example, subject set of FET-labeled substrates comprises a collection of from about 2 FET-labeled substrates to about 5 FET-labeled substrates, from about 5 FET-labeled substrates to about 10 FET-labeled substrates, from about 10 FET-labeled substrates to about 20 FET-labeled substrates, from about 20 FET-labeled substrates to about 25 FET-labeled substrates, from about 25 FET-labeled substrates to about 50 FET-labeled substrates, from about 50 FET-labeled substrates to about 75 FET-labeled substrates, or from about 75 FET-labeled substrates to about 100 FET-labeled substrates. In some embodiments, a subject set of FET-labeled substrates comprises more than 100 FET-labeled substrates.

Sets of FET-labeled substrates, e.g., sets of two or more, three or more, four or more, five or more, etc., FET-labeled substrates, comprise two or more FET-labeled substrates, each of which differs from the other in: i) nucleotide sequences of the substrate recognition domains (“recognition nucleotide sequence”); and ii) the FET labels, e.g., in the fluorescence donor and/or the fluorescence quencher.

The recognition nucleotide sequences of the FET-labeled substrates are complementary to recognition nucleotide sequences in the catalytic nucleic acid of the enzymatically active second nucleic acid amplification product. The recognition nucleotide sequences of the FET-labeled substrates are such that they hybridize only to the corresponding recognition nucleotide sequences in the catalytic nucleic acid of the enzymatically active second nucleic acid amplification product, e.g., the enzymatically active amplification product that comprises a nucleotide sequence of the target nucleic acid, and not to recognition nucleotide sequences in catalytic nucleic acids of other second nucleic acid amplification products.

The FET-labeled substrates in a set of FET-labeled substrates are distinguishable from one another, because, after being cleaved by the catalytic nucleic acid of the enzymatically active second nucleic acid amplification product, they fluoresce at different wavelengths that are distinguishable one from another. In a subject FET-labeled substrate set, any two FET-labeled substrates fluoresce, after being cleaved, at wavelengths that differ by from about 10 nm to about 400 nm, e.g., from about 10 nm to about 20 nm, from about 20 nm to about 30 nm, from about 30 nm to about 40 nm, from about 40 nm to about 50 nm, from about 50 nm to about 60 nm, from about 60 nm to about 70 nm, from about 70 nm to about 80 ml, from about 80 nm to about 90 nm, from about 90 nm to about 100 nm, from about 100 nm to about 125 nm, from about 125 nm to about 150 nm, from about 150 nm to about 175 nm, from about 175 nm to about 200 nm, from about 200 nm to about 250 m, from about 250 nm to about 300 nm, from about 300 nm to about 350 nm, or from about 350 nm to about 400 mm.

Thus, e.g., where two different target nucleic acids are being detected, a set of FET-labeled substrates includes: (a) a first FET-labeled substrate comprising a first recognition nucleotide sequence (e.g., a recognition domain R1 and a recognition domain R2, which are complementary to recognition domains R1 and R2, respectively, of a first catalytic nucleic acid); and further comprising a first fluorescence donor and a first fluorescence quencher; and (b) a second FET-labeled substrate comprising a second recognition nucleotide sequence (e.g., a recognition domain R3 and a second recognition domain R4, which are complementary to recognition domains R3 and R4, respectively, of a second catalytic nucleic acid); and further comprising a second fluorescence donor and a second fluorescence acceptor. The recognition nucleotide sequences of the first FET-labeled substrate are such that they do not hybridize to the recognition nucleotide sequences of the second catalytic nucleic acid; and the recognition nucleotide sequences of the second FET-labeled substrate are such that they do not hybridize to the recognition nucleotide sequences of the first catalytic nucleic acid. FIG. 3 is a schematic depiction of an exemplary duplex analysis, e.g., analysis of two different target nucleic acids using two different FET-labeled substrates.

As one non-limiting example, a set of FET-labeled substrates includes: (a) a first FET-labeled substrate comprising a first recognition nucleotide sequence; and further comprising FAM as a first fluorescence donor and BHQ as the fluorescence quencher; and (b) a second FET-labeled substrate comprising a second recognition nucleotide sequence; and further comprising JOE as a second fluorescence donor and BHQ as a second fluorescence acceptor.

In some embodiments, the fluorescent donor is a FRET pair. For example, in one embodiment, a set of FET-labeled substrates includes: (a) a first FET-labeled substrate comprising a first recognition nucleotide sequence; and further comprising FAM/TAMRA as a first fluorescence donor and BHQ as a first fluorescence quencher; and (b) a second FET-labeled substrate comprising a second recognition nucleotide sequence; and further comprising JOE as a second fluorescence donor and BHQ as a second fluorescence quencher.

As another non-limiting example, a set of FET-labeled substrates includes: (a) a first FET-labeled substrate comprising a first recognition nucleotide sequence; and further comprising JOE/TAMRA as a first fluorescence donor and BHQ as the fluorescence quencher; and (b) a second FET-labeled substrate comprising a second recognition nucleotide sequence; and further comprising TAMRA as a second fluorescence donor and BHQ as a second fluorescence acceptor.

As another non-limiting example, a set of FET-labeled substrates includes: (a) a first FET-labeled substrate comprising a first recognition nucleotide sequence; and further comprising TET-TAMRA as a first fluorescence donor and BHQ as the fluorescence quencher; and (b) a second FET-labeled substrate comprising a second recognition nucleotide sequence; and further comprising JOE as a second fluorescence donor and BHQ as a second fluorescence acceptor.

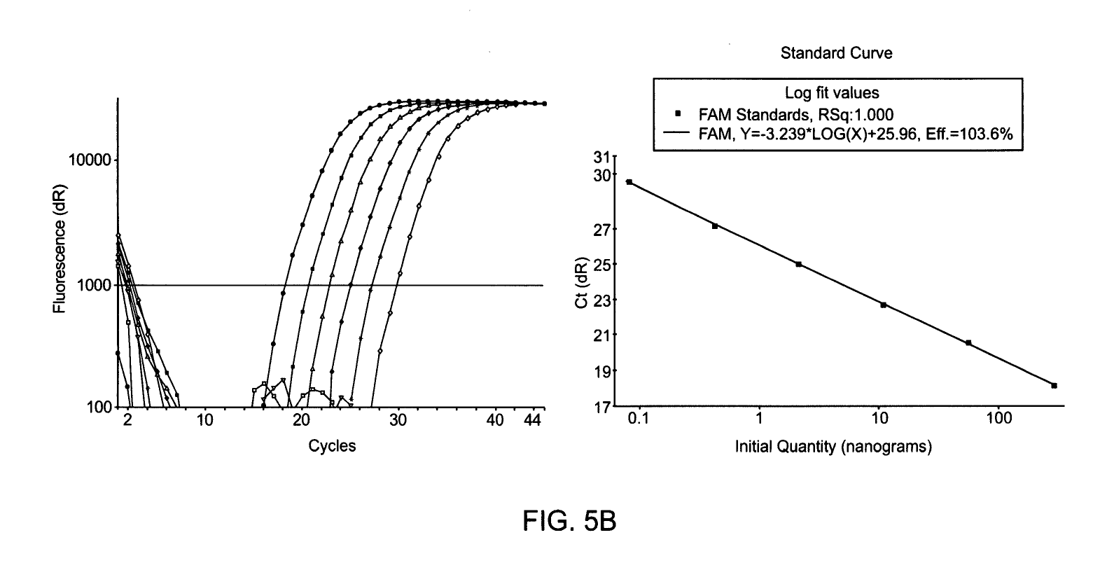

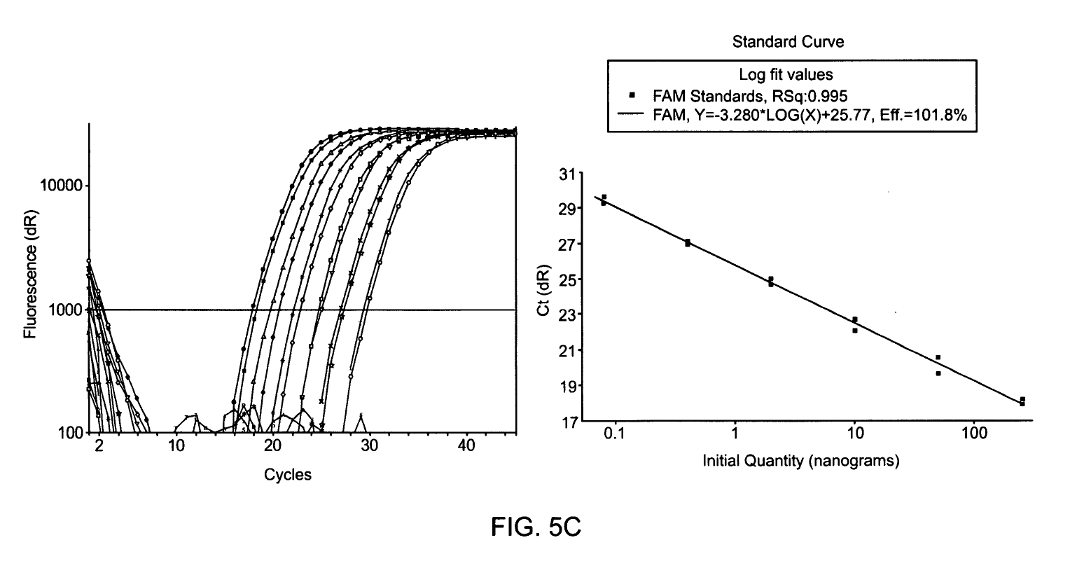

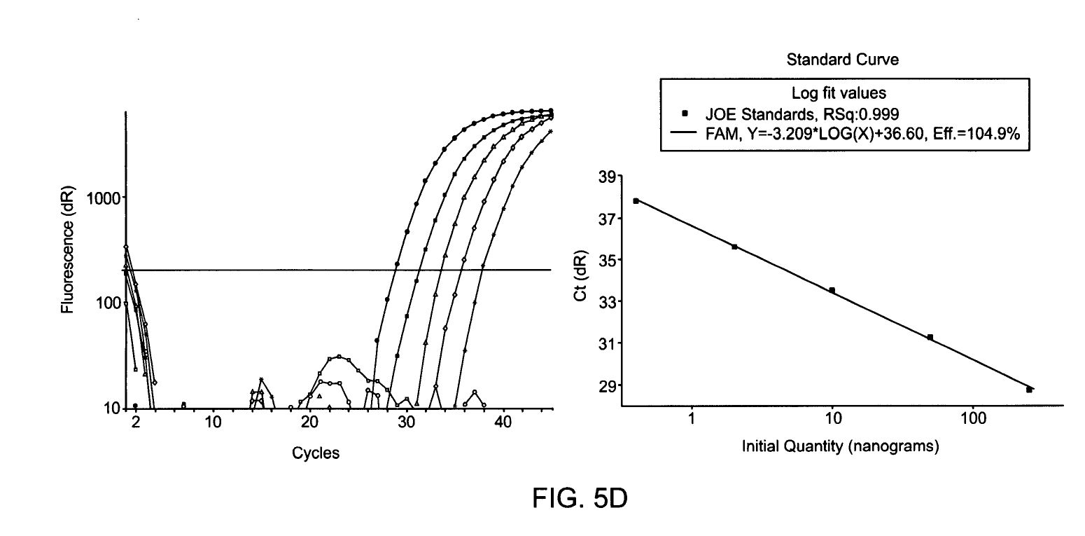

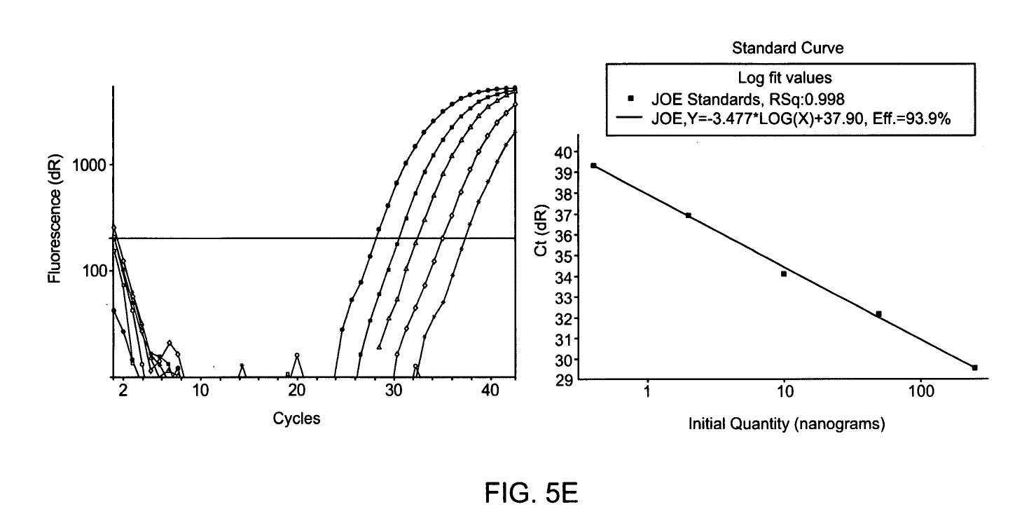

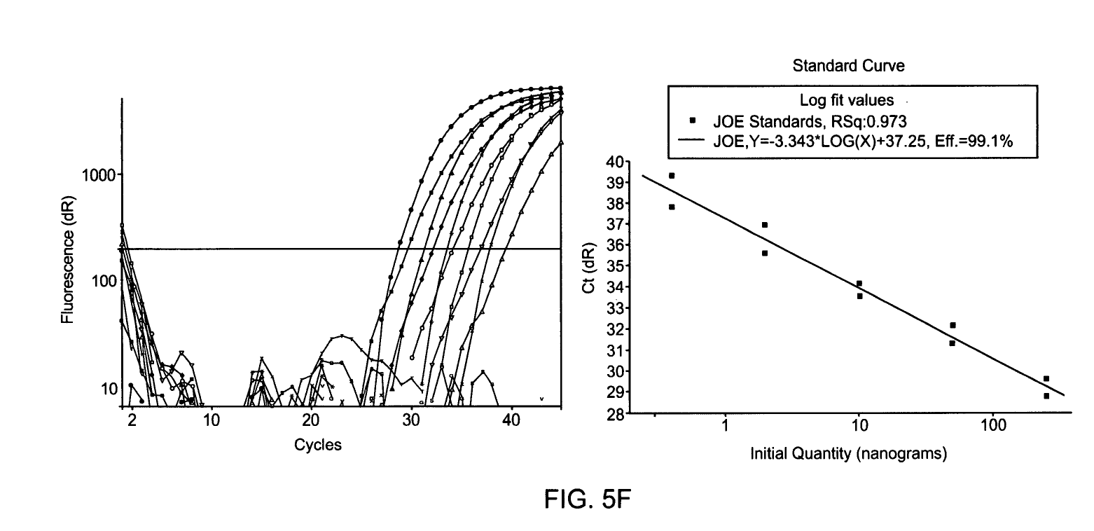

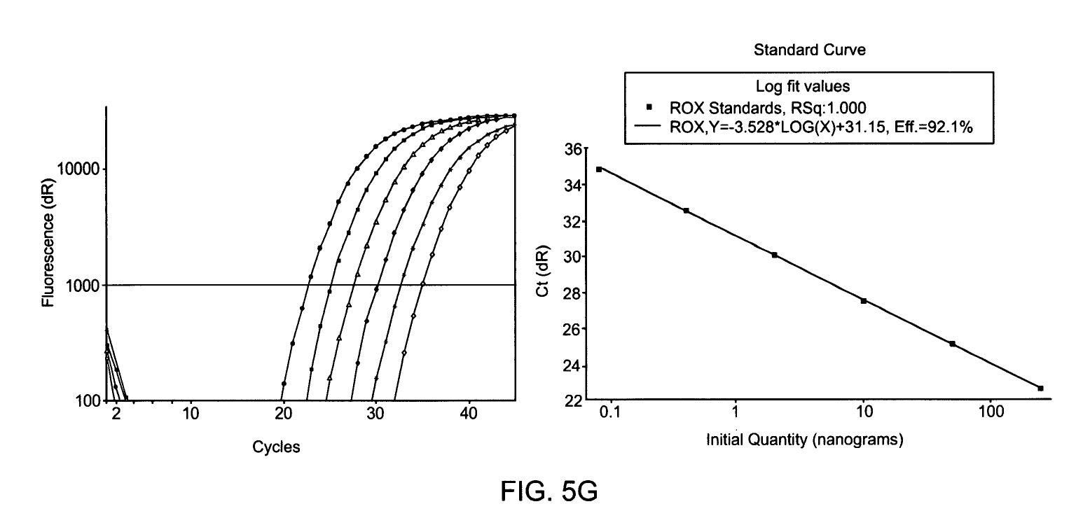

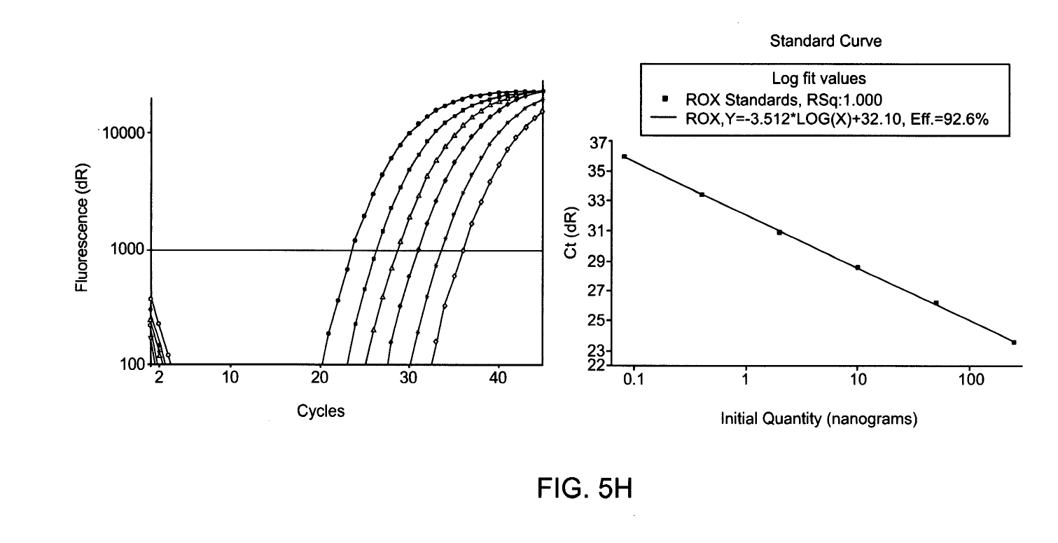

As another non-limiting example, a set of FET-labeled substrates includes: (a) a first FET-labeled substrate comprising a first recognition nucleotide sequence; and further comprising JOE/ROX as a first fluorescence donor and BHQ as the fluorescence quencher; and (b) a second FET-labeled substrate comprising a second recognition nucleotide sequence; and further comprising TAMRA as a second fluorescence donor and BHQ as a second fluorescence acceptor.

Where three different target nucleic acids are being detected, a set of FET-labeled substrates includes: (a) a first FET-labeled substrate comprising a first recognition nucleotide sequence (e.g., a recognition domain R1 and a recognition domain R2, which are complementary to recognition domains R1 and R2, respectively, of a first catalytic nucleic acid); and further comprising a first fluorescence donor and a first fluorescence quencher; (b) a second FET-labeled substrate comprising a second recognition nucleotide sequence (e.g., a recognition domain R3 and a recognition domain R4, which are complementary to recognition domains R3 and R4, respectively, of a second catalytic nucleic acid); and further comprising a second fluorescence donor and a second fluorescence acceptor; and (c) a third FET-labeled substrate comprising a third recognition nucleotide sequence (e.g., a recognition domain R5 and a recognition domain R6, which are complementary to recognition domains R5 and R6, respectively, of a third catalytic nucleic acid; and further comprising a third fluorescence donor and a third fluorescence acceptor. FIG. 5 is a schematic depiction of an exemplary triplex analysis, e.g., analysis of three different target nucleic acids using three different FET-labeled substrates.

As one non-limiting example, where three different target nucleic acids are being detected, a set of FET-labeled substrates includes: (a) a first FET-labeled substrate comprising a first recognition nucleotide sequence; and further comprising FAM as a first fluorescence donor and a BHQ as a first fluorescence quencher; (b) a second FET-labeled substrate comprising a second recognition nucleotide sequence; and further comprising JOE as a second fluorescence donor and a BHQ as a second fluorescence acceptor; and (c) a third FET-labeled substrate comprising a third recognition nucleotide sequence; and further comprising FAM/TAMRA as a third fluorescence donor and a BHQ as a third fluorescence acceptor.

As another non-limiting example, where three different target nucleic acids are being detected, a set of FET-labeled substrates includes: (a) a first FET-labeled substrate comprising a first recognition nucleotide sequence; and further comprising FAM as a first fluorescence donor and a BHQ as a first fluorescence quencher; (b) a second FET-labeled substrate comprising a second recognition nucleotide sequence; and further comprising JOE as a second fluorescence donor and a BHQ as a second fluorescence acceptor; and (c) a third FET-labeled substrate comprising a third recognition nucleotide sequence; and further comprising JOE/TAMRA as a third fluorescence donor and a BHQ as a third fluorescence acceptor.

As one non-limiting example, where three different target nucleic acids are being detected, a set of FET-labeled substrates includes: (a) a first FET-labeled substrate comprising a first recognition nucleotide sequence; and further comprising FAM as a first fluorescence donor and a BHQ as a first fluorescence quencher; (b) a second FET-labeled substrate comprising a second recognition nucleotide sequence; and further comprising JOE as a second fluorescence donor and a BHQ as a second fluorescence acceptor; and (c) a third FET-labeled substrate comprising a third recognition nucleotide sequence; and further comprising JOE/ROX as a third fluorescence donor and a BHQ as a third fluorescence acceptor.

Where four different target nucleic acids are being detected, a set of FET-labeled substrates includes: (a) a first FET-labeled substrate comprising a first recognition nucleotide sequence (e.g., a recognition domain R1 and a recognition domain R2, which are complementary to recognition domains R1 and R2, respectively, of a first catalytic nucleic acid); and further comprising a first fluorescence donor and a first fluorescence quencher; (b) a second FET-labeled substrate comprising a second recognition nucleotide sequence (e.g., a recognition domain R3 and a recognition domain R4, which are complementary to recognition domains R3 and R4, respectively, of a second catalytic nucleic acid); and further comprising a second fluorescence donor and a second fluorescence acceptor; (c) a third FET-labeled substrate comprising a third recognition nucleotide sequence (e.g., a recognition domain R5 and a recognition domain R6, which are complementary to recognition domains R5 and R6, respectively, of a third catalytic nucleic acid; and further comprising a third fluorescence donor and a third fluorescence acceptor; and (d) a fourth FET-labeled substrate comprising a fourth recognition nucleotide sequence (e.g., a recognition domain R7 and a recognition domain R8, which are complementary to recognition domains R7 and R8, respectively, of a fourth catalytic nucleic acid); and further comprising a fourth fluorescence donor and a fourth fluorescence quencher. FIG. 7 is a schematic depiction of an exemplary tetraplex analysis, e.g., analysis of four different target nucleic acids using four different FET-labeled substrates.

Other fluorescence donor/acceptor pairs are well known in the art; and any known fluorescence donor/acceptor pairs can be used. Non-limiting examples of suitable fluorescence donor/acceptor pairs are shown in Table 1.

TABLE 1

| | Excitation | Emission | |
|---|---|---|---|
| Fluorophore | maxima | maxima | |
| Donor | (nm) | (nm) | Suitable Acceptors |
| Fluorescein | 492 | 520 | TAMRA, ROX, Cy3, |
| | | | Cy3.5, Cy5, Cy6.6 |
| | | | DABCYL, BHQ-1/2, Eclipse |
| HEX | 535 | 556 | TAMRA, DABCL, BHQ-1/2, |
| | | | Eclipse ™ |
| JOE | 520 | 548 | TAMRA, DABCL, BHQ-1, |
| | | | ElleQuencher ™ |
| ROX | 585 | 605 | DABCYL, BHQ-2, |
| | | | ElleQuencher ™ |
| TAMRA | 565 | 605 | DABCYL, BHQ-2, |
| | | | ElleQuencher ™ |
| TET | 521 | 544 | TAMRA, DABCL, |
| | | | BHQ-1/2, Eclipse ™ |

In Table 1, HEX is (carboxy-2′,4,4′,5′,7,7′,hexachlorofluorescein); and Eclipse™ and ElleQuencher™ are dark quenchers.

The present invention provides compositions comprising a subject FET-labeled substrate set, where the compositions comprise a subject FET-labeled set and a second component. Suitable second components include one or more of a buffer, a nuclease inhibitor, a salt, etc. In some embodiments, a FET-labeled substrate set is provided as a liquid solution. In other embodiments, a FET-labeled substrate set is lyophilized.

Sets of Zymogenes

The present invention further provides sets of zymogenes (e.g., nucleic acids that comprise both a zymogene and a target-specific nucleic acid primer), which are used in conjunction with a set of FET-labeled substrates with corresponding recognition nucleotide sequences. A subject set of zymogenes includes two or more zymogenes, such that for each target being detected, there exists at least one zymogene which encodes, but which itself is the anti-sense sequence of, a catalytic nucleic acid sequence having distinctly measurable activity, where each zymogene comprises a different 5′ primer that initiates synthesis of a different first, enzymatically inactive nucleic acid amplification product that comprises a nucleotide sequence that is complementary to a different target nucleic acid.

A subject set of zymogenes includes at least two, at least three, at least four, at least five, at least six, at least seven, at least eight, at least nine, or at least ten different zymogenes, each with a different recognition nucleotide sequence that is substantially identical to a recognition nucleotide sequence in a corresponding FET-labeled substrate; and each with a different, target-specific nucleic acid primer.

Thus, e.g., a subject set of zymogenes includes: (a) a first nucleic acid comprising a first recognition sequence that is the same as a recognition sequence in a first FET-labeled substrate (e.g., a recognition domain R1 and a recognition domain R2, which are substantially identical to recognition domains R1 and R2, respectively, of a first FET-labeled substrate); and further comprising a first nucleic acid primer that is complementary to and hybridizes to a nucleotide sequence in a first target nucleic acid; and (b) a second nucleic acid comprising a second recognition sequence that is the same as a recognition sequence in a second FET-labeled substrate (e.g., a recognition domain R3 and a recognition domain R4, which are substantially identical to recognition domains R3 and R4, respectively, of a second FET-labeled substrate; and further comprising a second nucleic acid primer that is complementary to and hybridizes to a nucleotide sequence in a second target nucleic acid.

In some embodiments, a subject set of zymogenes includes: (a) a first nucleic acid comprising a first recognition sequence that is the same as a recognition sequence in a first FET-labeled substrate (e.g., a recognition domain R1 and a recognition domain R2, which are substantially identical to recognition domains R1 and R2, respectively, of a first FET-labeled substrate); and further comprising a first nucleic acid primer that is complementary to and hybridizes to a nucleotide sequence in a first target nucleic acid; (b) a second nucleic acid comprising a second recognition sequence that is the same as a recognition sequence in a second FET-labeled substrate (e.g., a recognition domain R3 and a recognition domain R4, which are substantially identical to recognition domains R3 and R4, respectively, of a second FET-labeled substrate; and further comprising a second nucleic acid primer that is complementary to and hybridizes to a nucleotide sequence in a second target nucleic acid; and (c) a third nucleic acid comprising a third recognition sequence that is the same as a recognition sequence in a third FET-labeled substrate (e.g., a recognition domain R5 and a recognition domain R6, which are substantially identical to recognition domains R5 and R6, respectively, of a third FET-labeled substrate; and further comprising a third nucleic acid primer that is complementary to and hybridizes to a nucleotide sequence in a third target nucleic acid.

Utility

The subject methods and compositions are useful in a variety of applications, including experimental applications and diagnostic applications. The subject methods are useful for gene expression analysis; analytical polymerase chain reaction assays; transgene quantification; insert screening; viral load determinations; and the like.

In one embodiment, the instant method is used for diagnostic purposes. Specifically, the invention can be used to diagnose a disorder in a subject characterized by the presence of at least one target nucleic acid sequence which is not present when such disorder is absent. Such disorders are well known in the art and include, by way of example, cancer, cystic fibrosis, and various hemoglobinopathies. The invention can also be used to diagnose disorders associated with the presence of infectious agents. Such disorders include, by way of example, acquired immunodeficiency syndrome, Hepatitis C virus infection, and tuberculosis. In an exemplary embodiment, the subject being diagnosed is human and the disorder is cancer.

One type of representative application is in monitoring the progress of nucleic acid amplification reactions, such as polymerase chain reaction applications, including both linear and geometric PCR applications. As used herein, the term monitoring includes a single evaluation at the end of a series of reaction cycles as well as multiple evaluations, e.g., after each reaction cycle, such that the methods can be employed to determine whether a particular amplification reaction series has resulted in the production of primer extension product, e.g., a non-real time evaluation, as well as in a real-time evaluation of the progress of the amplification reaction.

The subject methods find use in both 5′ nuclease methods of monitoring a PCR amplification reaction (e.g., where a Taqman type probe is employed); and non-5′ nuclease methods of monitoring a PCR amplification reaction (e.g., where a molecular beacon type probe is employed). Again, the subject methods find use in evaluating the progress of an amplification reaction at a single time (e.g., non-real time monitoring) and in real-time monitoring.

Monitoring a PCR reaction according to the subject methods finds use in a variety of specific applications. Representative applications of interest include, but are not limited to: (1) detection of allelic polymorphism; (2) single nucleotide polymorphism (SNP) detection; (3) detection of rare mutations; (4) detection of allelic stage of single cells; (5) detection of single or low copy number DNA analyte molecules in a sample; etc. For example, in detection of allelic polymorphism, a nucleic acid sample to be screened, e.g., a genomic DNA cellular extract, is employed as template (target) nucleic acid in the preparation of a primer extension reaction mixture, as described above, where the reaction mixture includes a different and distinguishable FET-labeled substrates that is specific for each different allelic sequence to be identified, if present. The assay is then carried out as described above, where the sample is screened for a change in signal from each different FET-labeled substrate. A change in signal from a given FET-labeled substrate is indicative of the presence the allelic variant to which that FET-labeled substrate is specific in the sample. Likewise, an absence of change in signal is indicative of the absence of the allelic variant in the sample. In this manner, the sample is readily screened for the presence of one or more allelic variants. A similar approach can be used for SNP detection, where a different FET-labeled substrate for each SNP of interest to be screened in a nucleic acid sample is employed.

Kits

This invention still further provides a kit for use in determining the presence of one or more target nucleic acid sequences in a sample.

In some embodiments, a subject kit comprises one or more of:
a) a first nucleic acid, wherein the first nucleic acid comprises a DNA zymogene which encodes, but which itself is the anti-sense sequence of, a catalytic nucleic acid; and a first primer that initiates synthesis of a first, enzymatically inactive nucleic acid amplification product that comprises a nucleotide sequence that is complementary to the target nucleic acid;
(b) a second primer nucleic acid that hybridizes to the first amplification product and initiates synthesis of a second, enzymatically active nucleic acid amplification product, which comprises a nucleotide sequence of the target nucleic acid, and the catalytic nucleic acid molecule;
(c) a third nucleic acid that comprises a nucleotide sequence that is complementary to the target nucleic acid, which nucleotide sequence at least partially overlaps with the nucleotide sequence of the first nucleic acid primer; and
(d) a FET-labeled nucleic acid substrate, where the FET-labeled nucleic acid substrate comprises a recognition nucleotide sequence that is complementary to a recognition nucleotide sequence present in the catalytic nucleic acid; and a cleavage site that is cleaved by the catalytic nucleic acid.

Where the kit includes a zymogene and a third nucleic acid (e.g., a “driver primer”), in many embodiments the driver primer is present in a molar ratio, relative to the zymogene, of 1:1 or greater, e.g., 1.5:1, 2:1, 2.5:1, 3:1, or greater. In many of these embodiments, the third nucleic acid comprises a 3′ OH modification such that the third nucleic acid does not initiate, or prime, synthesis of a nucleic acid amplification product.

In other embodiments, a subject kit comprises one or more of:
(a) at least one zymogene which zymogene comprises a DNA zymogene and a first nucleic acid primer that initiates synthesis of a first, enzymatically inactive nucleic acid amplification product (e.g., a first, enzymatically inactive nucleic acid extension product) that comprises a nucleotide sequence that is complementary to the target nucleic acid;
(b) at least one second primer nucleic acid that hybridizes to the first amplification product (e.g., the first, enzymatically inactive extension product) and initiates synthesis of a second, enzymatically active nucleic acid amplification product (e.g., a second, enzymatically active nucleic acid extension product), which comprises a nucleotide sequence of the target nucleic acid, and the catalytic nucleic acid molecule;
(c) reagents permitting primer-initiated nucleic acid amplification and catalytic nucleic acid activity; and
(d) a set of one or more, e.g., two or more, FET-labeled substrates.

In some embodiments, a subject kit further comprises a plurality of driver primers, where, for each target nucleic acid being detected, there is a corresponding driver primer present in the kit.

In some embodiments, a subject kit comprises a subject FET-labeled oligonucleotide substrate set. In some embodiments, a subject kit comprises a subject zymogene set. In some embodiments, a subject kit includes both a subject FET-labeled oligonucleotide substrate set and a subject zymogene set.

Reagents permitting primer-initiated nucleic acid amplification and catalytic nucleic acid activity include one or more of the following: a) a set of dNTPs; b) magnesium ions, e.g., MgCl₂; c) a DNA polymerase, as described above; etc.

In one embodiment, the instant kit further comprises reagents useful for isolating a sample of nucleic acid molecules from a subject or sample. The components in the instant kit can either be obtained commercially or made according to well known methods in the art, as exemplified in the Examples section below. In addition, the components of the instant kit can be in solution or lyophilized as appropriate. In one embodiment, the components are in the same compartment, and in another embodiment, the components are in separate compartments. In many embodiments, the kit further comprises instructions for use.

In the instant methods and kits, the nucleic acid amplification can be performed according to any suitable method known in the art, and preferably according to one selected from the group consisting of PCR, SDA and TMA.

The various reagent components of the kits may be present in separate containers, or may all be precombined into a reagent mixture for combination with template (e.g., target) DNA. For example, the kit may include a set of FET-labeled substrates and a corresponding set of zymogenes, where these two components may be present separately or combined into a single composition for use.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, compact disk (CD), etc., on which the information has been recorded. Yet another means that may be present is a website address which may be used via the internet to access the information at a removed site. Any convenient means may be present in the kits.

Systems

Also provided are systems for use in practicing the subject methods. The subject systems at least include one or more FET-labeled oligonucleotide substrates, as well as any other requisite components for practicing the subject methods, as described above. In addition, the subject systems may include any required devices for practicing the subject methods, e.g., thermal cyclers, fluorimeters, etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); nt, nucleotide(s); i.m., intramuscular(ly); i.p., intraperitoneal(ly); s.c., subcutaneous(ly); and the like.

### Example 1

### Multiplex Quantitative PCR

To achieve a multiplex assay (duplex, triplex and tetraplex); a combination of reporter substrates were used. For example, in a duplex assay, a FAM and a JOE reporter substrates were used; in a triplex assay, a FAM, a JOE and a FAM-TAM (or a ROX or a Cy5) reporter substrates were used; in a tetraplex assay, a FAM, a JOE, a ROX and a Cy5 reporter substrates were used. In addition, each individual reporter substrate with a unique fluorescent dye was recognized and cleaved by an active DNAzyme that was generated from PCR reaction from zymogene sequence. The zymogene sequence contained antisense catalytic domain of DNAzyme flanked by two sequences that determines the reporter substrate specificity.

Duplex Analysis

Two gene targets were assayed in a duplex format. To evaluate the duplex assay performance, the real-time PCR plots and standard curves of each gene in single-plex and duplex assays were compared. The experiment was carried out on a Stratagene MX3000p instrument. The target gene and primer information are provided in Table 2, below.

TABLE 2

| | GenBank | Gene specific | Gene specific |
|---|---|---|---|
| Gene | Accession | forward primers | reverse primers |
| Symbol | No. | 5′->3′ | 5′->3′ |
| Bmp4 | X56848 | CTCCCAAGAATCATGGACTG | AAAGCAGAGCTCTCACTGGT |
| | | SEQ ID NO: 18 | SEQ ID NO: 19 |
| Actb | NM_007393 | ACCCCTAAGGCCAACCGTG | CAGGATTCCATACCCAAGAAGG |
| | | SEQ ID NO: 20 | SEQ ID NO: 21 |

Triplex Analysis

Three gene targets are assayed in a triplex format. To evaluate the triplex assay performance, the real-time PCR plots and standard curves of each gene in single-plex and triplex assays were compared. The experiment was carried out on a Stratagene MX3000p instrument. Specifically, serial 5-fold dilutions of human universal cDNA (template) were added to a master mix containing the appropriate primer pair(s), PCR buffer, QTaq PCR Polymerase and water. PCR was performed under the following conditions: 1 cycle of: 95° C. for 3 minutes; followed by 45 cycles of: 95° C. for 15 seconds; then 56° C. for 1 minute. Amplification plots were collected for the appropriate fluorescent channels (depending on primers/substrates used) and graphed as Fluorescence (dR) versus cycle number. The threshold cycle (Cₜ) was calculated by setting the fluorescence at 1000 units (arbitrary scale). Cₜ values were then graphed against the amount of human universal cDNA (template) on a semi-log plot to generate the standard curves seen on the right hand panel of each figure. The slopes of the standard curves give an indication of the PCR efficiency whereas the correlation coefficient of the linear regression through the data indicates the quality of the data thus generated.

The target gene and primer information are provided in Table 3, below.

TABLE 3

| | GenBank | Gene specific | Gene specific |
|---|---|---|---|
| Gene | Accession | forward primers | reverse primers |
| Symbol | No. | 5′->3′ | 5′->3′ |
| Colla2 | NM_007743 | GGCAAGACAATCATTGAA | GGTTGAGTTCACTTATTTGAA |
| | | SEQ ID NO: 22 | SEQ ID NO: 23 |
| Mmp13 | NM_008607 | ATGACCTATAGACTCTTTGA | CTGTCACACAAGTTACTAGA |
| | | SEQ ID NO: 24 | SEQ ID NO: 25 |
| Rp113 | BC055358 | TTTTGCCAGTCTCCGAAT | TGCTTTATGGAAAATTTATTGC |
| | | SEQ ID NO: 26 | SEQ ID NO: 27 |

The results are depicted in FIGS. 5A-I. For each gene in this triplex assay, data are shown from the single-plex assay (FIGS. 5a, Col1a2; 5d, Mmp13; and 5g, Rpl13) and the triplex assay (all 3 primer pairs present) (FIGS. 5b, Col1a2; 5e, Mmp13; and 5h, Rpl13) in each channel. Also shown is the overlap of the two data sets (FIGS. 5c, Col1a2; 5f, Mmp13; 5i, Rpl13), which shows that the assay performance in each channel is not affected by the presence of the other primer pairs. These data can be represented by delta (Ct) shift, which ideally is 0, and in this case is less than 1 cycle difference between the single- and triplex assays. Triplex efficacy is also assessed by having slopes of the standard curves indicative of good PCR efficiency (>90%) and correlation coefficients >0.990.

Tetraplex Analysis

In the following example, four genes target assayed simultaneously in a tetraplex format on a Stratagene MX3000p instruments.

The four gene target targets are depicted in Table 4, below, along with the forward primer (5′ primer) and reverse primer (3′ primer) that amplify each.

TABLE 4

| | GenBank | Gene specific | Gene specific |
|---|---|---|---|
| Gene | Accession | forward primers | reverse primers |
| Symbol | No. | 5′->3′ | 5′->3′ |
| RRN18S | X03205 | GGTCTGTGATGCCCTTAGA | AGCTTATGACCCGCACTTA |
| | | SEQ ID NO: 28 | SEQ ID NO: 29 |
| RPLP0 | NM_001002 | CGACCTGGAAGTCCAACTA | TTCGGATAATCATCCAATAG |
| | | SEQ ID NO: 30 | SEQ ID NO: 31 |
| GAPD | NM_002046 | CAACGGATTTGGTCGTATT | TTGATGGCAACAATATCCA |
| | | SEQ ID NO: 32 | SEQ ID NO: 33 |
| B2M | NM_004048 | TCGCGCTACTCTCTCTTTC | ATCTTTGGAGTACGCTGGA |
| | | SEQ ID NO: 34 | SEQ ID NO: 35 |

In a real-time PCR assay, the forward primers (5′ primers) were included in nucleic acids that also included a zymogene sequence containing the antisense catalytic domain of the DNAzyme flanked by two sequences (e.g., R1 and R2, as shown schematically in FIG. 2) that determine the reporter substrates' specificity. In this example, FAM was used to report RRN18S gene expression level, JOE to report RPLP0 gene expression level, TAMRA to report GAPD gene expression level and finally, ROX to report B2M gene expression level.

5′ Driver Primer

A single gene target (SOX9) was used to assess the addition of a 5′ “driver” primer to the real-time PCR assay. PCR reactions contained a primer pair designed to amplify the target, and included PCR buffer, QTaq PCR enzyme and water. Amplification and data analysis was accomplished as detailed above in the description of the triplex analysis. The gene target tested was SOX9, GenBank Accession No. NM_{—}000346. The forward gene specific primer had the sequence: 5′ ATGTGTCATCCATATTTCTC 3′ (SEQ ID NO:36); and the reverse primer had the sequence: 5′ CCACAGCAGTAATTAAGATT 3′ (SEQ ID NO:37).

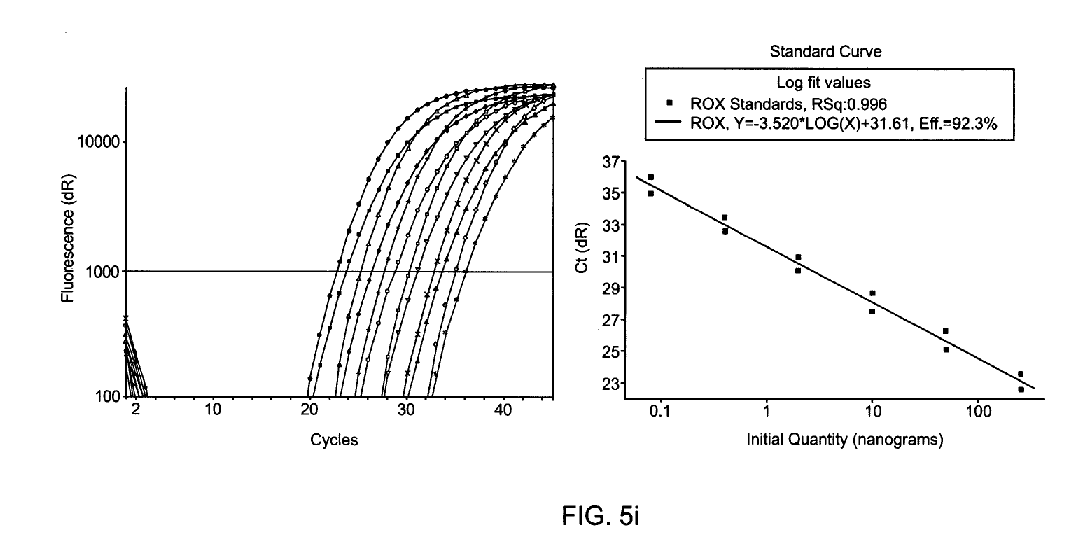

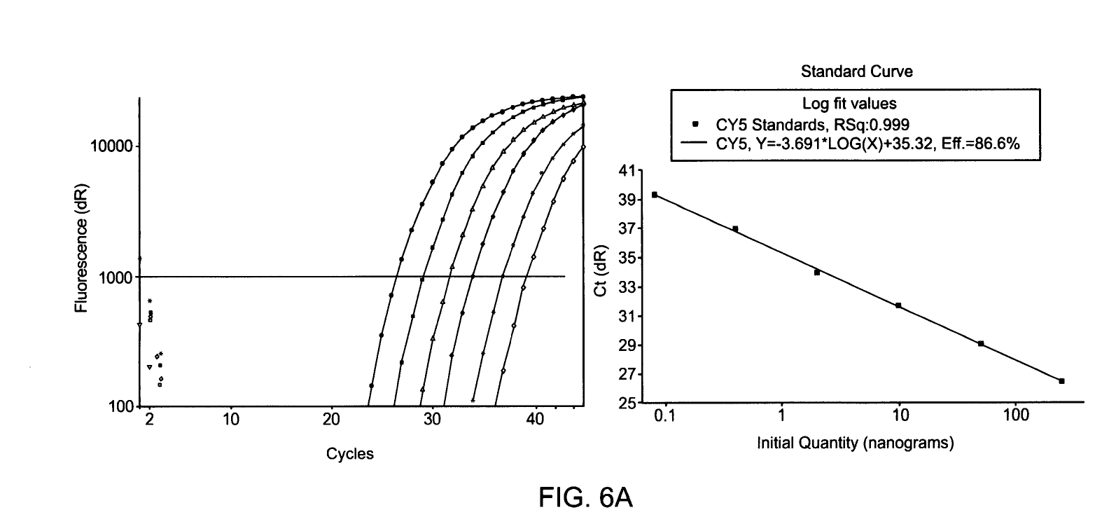

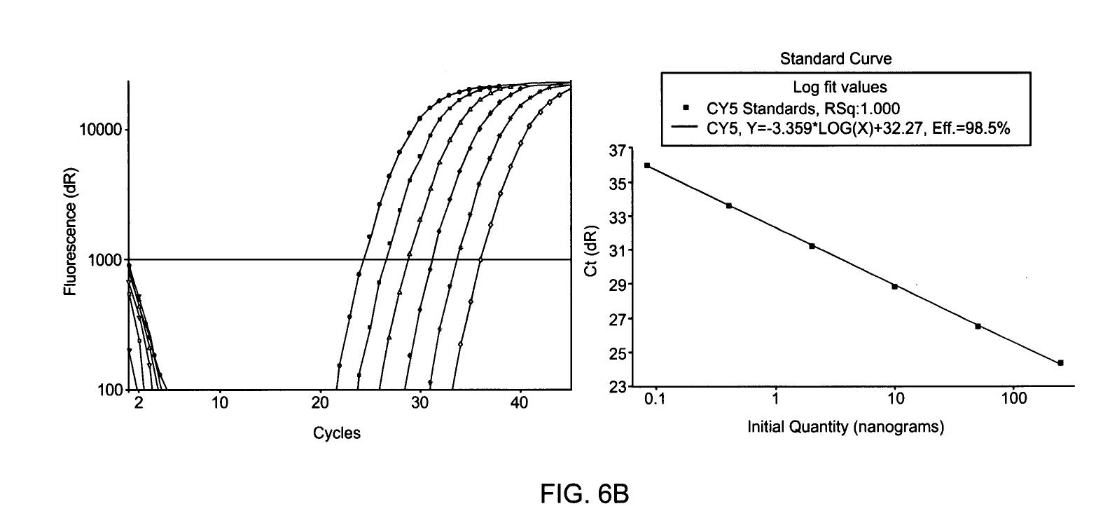

The data are shown in FIGS. 6A and 6B. In the absence of primer driver, the PCR efficiency was determined to be 86.6%. Upon addition of primer driver (equimolar to the forward primer at 60 nM), the analyzed data showed an increase of PCR efficiency to 98.5% in this single-plex assay.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for detecting the presence of a target nucleic acid in a sample, the method comprising:
(a) contacting the sample with:
(i) a first nucleic acid, wherein the first nucleic acid comprises a DNA zymogene and a first primer that initiates synthesis of a first, enzymatically inactive nucleic acid amplification product that comprises a nucleotide sequence that is complementary to the target nucleic acid;
(ii) a second primer nucleic acid that hybridizes to the first amplification product and initiates synthesis of a second, enzymatically active nucleic acid amplification product, which comprises a nucleotide sequence of the target nucleic acid, and the catalytic nucleic acid molecule; and
(iii) a third nucleic acid that comprises a nucleotide sequence that is complementary to the target nucleic acid, which nucleotide sequence at least partially overlaps with the nucleotide sequence of the first nucleic acid primer; and
(b) detecting the presence of catalytic nucleic acid activity, wherein the presence of catalytic nucleic acid activity indicates the presence of the target nucleic acid in the sample.

2. The method of claim 1, wherein the third nucleic acid is present in the sample at a molar ratio of at least about 1:1 with the first nucleic acid.

3. The method of claim 1, wherein the third nucleic acid is present in the sample at a molar ratio of at least about 1.5:1 with the first nucleic acid.

4. The method of claim 1, wherein the third nucleic acid is present in the sample at a molar ratio of at least about 2:1 with the first nucleic acid.

5. The method of claim 1, wherein the third nucleic acid comprises a 3′ OH modification such that the third nucleic acid does not initiate synthesis of a nucleic acid amplification product.

6. The method of claim 1, wherein the second nucleic acid primer does not yield an enzymatically active nucleic acid amplification product in a control sample lacking a target nucleic acid.

7. A method of detecting the presence of a plurality of target nucleic acid sequences in a sample, the method comprising:
(a) contacting the sample with:
(i) a plurality of zymogenes, wherein for each target being detected, there exists at least one zymogene, wherein each zymogene comprises a different first primer that initiates synthesis of a different first, enzymatically inactive nucleic acid amplification product that comprises a nucleotide sequence that is complementary to a different target nucleic acid; and
(ii) a plurality of second nucleic acid primers wherein for each target being detected, there exists at least one second nucleic acid primer that hybridizes to the first amplification product and initiates synthesis of a second, enzymatically active nucleic acid amplification product, which comprises a nucleotide sequence of the corresponding target nucleic acid, and the catalytic nucleic acid;
(b) detecting the presence of each of the catalytic nucleic acid activities by employing a separate fluorescent energy transfer-labeled (FET-labeled) substrate for each of the catalytic activities, wherein at least one of the FET-labeled substrates include a dark quencher;
thereby determining the presence of each of the corresponding-target nucleic acid sequences in the sample.

8. The method according to claim 7, wherein said plurality comprises at least two different target nucleic acids.

9. The method according to claim 7, wherein said plurality comprises at least 3 different target nucleic acids.

10. The method according to claim 7, wherein said plurality comprises at least 4 different target nucleic acids.

11. The method according to claim 7, wherein all of said FET labeled substrates comprise a dark quencher.

12. The method according to claim 7, wherein at least one of said FET labeled substrates comprises a fluorescence resonance energy transfer (FRET) pair at a first terminus and an acceptor at a second terminus.

13. The method according to claim 12, wherein said acceptor is a dark quencher.

14. The method of claim 7, further comprising the step of quantitatively determining the amount of each catalytic nucleic acid activity in the sample resulting from step (a), and comparing the amount of each activity so determined to a known standard, thereby quantitatively determining the amount of each target nucleic acid sequence.

15. The method of claim 7, wherein the target nucleic acid sequences are DNA molecules.

16. The method of claim 7, wherein the catalytic nucleic acid molecules are DNAzymes.

17. The method of claim 7, wherein step (a) further comprises contacting the sample with a plurality of third nucleic acids, each of which comprises a nucleotide sequence that is complementary to a target nucleic acid, which nucleotide sequence at least partially overlaps with the nucleotide sequence one of the first nucleic acid primers.

18. The method of claim 17, wherein the each of the third nucleic acids is present in the sample at a molar ratio of at least about 1:1 with the corresponding zymogene.

19. The method of claim 17, wherein at least one of the third nucleic acids comprises a 3′ OH modification such that the third nucleic acid does not initiate synthesis of a nucleic acid amplification product.

20. The method of claim 7, wherein at least one of the second nucleic acid primers does not yield an enzymatically active nucleic acid amplification product in a control sample lacking a target nucleic acid.

21. A composition comprising:
a) a plurality of fluorescent energy transfer-labeled (FET-labeled) nucleic acid substrates, wherein each of the FET-labeled nucleic acid substrates comprises: i) a recognition nucleotide sequence that is complementary to a recognition nucleotide sequence present in a catalytic nucleic acid; ii) a substrate cleaved by the catalytic nucleic acid; iii) a fluorescence donor at a first end of the nucleic acid substrate; and iv) a fluorescence acceptor at a second end of the nucleic acid substrate; and
b) a buffer.

22. The composition of claim 21, wherein at least one of the FET-labeled substrates comprises a dark quencher.

23. The composition of claim 21, wherein a first FET-labeled nucleic acid substrate comprises 5-carboxyfluorescein at the first end and a dark quencher at the second end; and wherein a second FET-labeled nucleic acid substrate comprises 2′,7′-dimethoxy-4′,5′-dichloro-6-carboxyfluorescein at the first end and a dark quencher at the second end.

24. A composition comprising:
a) a plurality of zymogenes, wherein each zymogene comprises: i) a zymogene domain; and ii) a different first primer nucleic acid that initiates synthesis of a different first, enzymatically inactive nucleic acid amplification product that comprises a nucleotide sequence that is complementary to a different target nucleic acid; and
b) a buffer.

25. A kit for use in determining the presence of a plurality of target nucleic acid sequences in a sample, which kit comprises:
(a) a plurality of zymogenes, wherein each zymogene comprises: i) a zymogene domain; and ii) a different first primer that initiates synthesis of a different first, enzymatically inactive nucleic acid amplification product that comprises a nucleotide sequence that is complementary to a different target nucleic acid;
(b) a plurality of primers, wherein for each target being detected, there exists at least one primer suitable for initiating amplification of that target;
(c) reagents permitting primer-initiated nucleic acid amplification and catalytic nucleic acid activity; and
d) a set of fluorescent energy transfer (FET)-labeled substrates comprising a substrate for each of the catalytic activities corresponding the plurality of zymogenes, wherein at least one of the FET-labeled substrates include a dark quencher.

26. The kit according to claim 25, wherein said set comprises at least two different substrates.

27. The kit according to claim 25, wherein said set comprises at least 3 different substrates.

28. The kit according to claim 25, wherein said set comprises at least 4 different substrates.

29. The kit according to claim 25, wherein all of said FET-labeled substrates of said set comprise a dark quencher.

30. The kit according to claim 25, wherein at least one of said FET-labeled substrates comprises a fluorescent resonance energy transfer pair at a first terminus and an acceptor at a second terminus.

31. The kit according to claim 30, wherein said acceptor is a dark quencher.
